(19) United States
(12) Patent Application Publication
(10) Pub. No.: US 2011/0189308 A1
(43) Pub. Date: Aug. 4, 2011

(54) METHODS TO TREAT NEURODEGENERATIVE CONDITIONS OR DISEASES BY TARGETING COMPONENTS OF A PTEN SIGNALING PATHWAY

(76) Inventors: Asa Abeliovich, New York, NY (US); Keiichi Inoue, New York, NY (US)

(21) Appl. No.: 12/933,186

(22) PCT Filed: Mar. 17, 2009
(86) PCT No.: PCT/US09/37354

§ 371 Date: Apr. 19, 2011

Related U.S. Application Data
(60) Provisional application No. 61/037,178, filed on Mar. 17, 2008.

(51) Int. Cl. A61K 33/14; A61K 31/407; A61K 31/425; A61K 31/404; A61K 31/55; A61K 31/505; A61K 31/426; A61K 31/506; A61K 31/7052; A61P 25/00

(52) U.S. Cl. 424/677; 514/411; 514/365; 514/415; 514/213.01; 514/256; 514/418; 514/215; 514/414; 514/371; 514/275; 514/44 R

(57) ABSTRACT

The disclosure provides that components of a PTEN cell signaling pathway may be utilized as therapeutic targets for treating, preventing, slowing the progression of or delaying the onset of a neurodegenerative disease or disorder. Such components that may be targeted include, phosphatase and tensin homologue (PTEN), glycogen synthase kinase 3 beta (GSK3β), and AKT. The subject matter disclosed herein relates to the therapeutic use of inhibitors of PTEN, inhibitors of GSK3β, or activators of AKT to treat a neurodegenerative disease or disorder in a subject. Compounds are disclosed which may be used in the methods provided.

This application claims priority to U.S. Provisional Application No. 61/037,178 filed on Mar. 17, 2008, which is hereby incorporated by reference in its entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Parkinson's disease is the second most common neurodegenerative disease, typically presenting as a progressive movement disorder with slowness, rigidity, gait difficulty, and tremor at rest. The pathological hallmarks of PD include the loss of dopamine (DA) neurons in the substantia nigra (SN) of the ventral midbrain and the presence of intracytoplasmic protein aggregates, termed Lewy bodies, composed of the synaptic vesicle-associated protein αSynuclein (αSyn), ubiquitin, and other components. It is thought that the earliest pathological feature of PD is the loss of dopaminergic axonal processes that extend from the substantia nigra to the striatum, preceding the eventual loss of DA neuron cell bodies. PD pathology has been described broadly in the CNS and is not confined to midbrain DA neurons.

Molecular clues regarding the etiology of the disease were lacking until the identification of genes that underlie familial, inherited forms of Parkinsonism. Missense mutations and duplications in αSyn are associated with rare cases of autosomal dominant familial Parkinsonism. αSyn mutations lead to increased aggregation of the protein as well as altered vesicular trafficking and defective protein degradation through proteasome and lysosome pathways. The presence of αSyn aggregates in LB inclusions that typify sporadic PD support the notion that familial forms of Parkinsonism are informative with respect to the mechanism of sporadic PD. Mutations in Parkin, DJ-1, and Pink1 lead to autosomal recessive Parkinsonism and are associated with increased sensitivity to oxidative stress as well as mitochondrial dysfunction, further implicating these mechanisms in Parkinsonism. Autosomal dominant mutations in leucine-rich repeat kinase-2 (LRRK2, PARK8, dardarin; OMIM 609007) were described in a familial Parkinsonism syndrome that mimics the clinical and pathological features of the common, sporadic form of PD.

To make significant progress towards treating neurodegenerative diseases such as PD, Alzheimer's disease and amyotrophic lateral sclerosis, it is important to identify molecular and genetic targets which can be used to develop new therapeutic compounds and treatment strategies.

### SUMMARY

A method is provided for treating a neurodegenerative disease or condition in a subject, the method comprising administering to the subject an effective amount of a compound that activates a phosphoinositide-3 kinase (PI3K) pathway, wherein the compound comprises one or more compounds selected from the group consisting of: an inhibitor of PTEN, an inhibitor of GSK3β and an activator of AKT.

In one embodiment, the treating comprises slowing progression of the neurodegenerative disease or condition. In one embodiment, the neurodegenerative disease comprises Parkinson's disease, Alzheimer's disease or amyotrophic lateral sclerosis. In one embodiment, the Parkinson's disease comprises mutation of a LRRK2 protein in the subject. In one embodiment, the neurodegenerative disease or disorder comprises deficient autophagy in neurons of the subject. In one embodiment, the administering comprises direct administration to the subject's brain.

In one embodiment, the inhibitor of PTEN comprises a vanadium complex. In one embodiment, the inhibitor of PTEN comprises one or more compounds selected from the group consisting of: VO—OHpic, bpV—OHpic, pbV-pic, VO-pic, bpV-biguan, VO-biguan, bpV-phen and bpV-isoqu. In one embodiment, the inhibitor of GSK3β comprises an ion, an arylindolemaleimide, a thiazole, a bis-indole, a benzazepinone or an aminopyrimidine.

In one embodiment, the inhibitor of GSK3β comprises one or more compounds selected from the group consisting of: indirubin-3′-monoxime, alsterpaullone, kenpaullone, SB216763, AR-A014418, CHIR98014 and lithium chloride.

In one embodiment, the activator of AKT comprises a plasmid capable of expressing an AKT protein, or a fragment thereof, from a nucleic acid encoding the AKT protein, or fragment thereof.

The issued patents, applications, and other publications that are cited herein are hereby incorporated by reference to the same extent as if each was specifically and individually indicated to be incorporated by reference.

### BRIEF DESCRIPTION OF THE FIGURES

FIGS. 1A-1E. Progressive loss of dopaminergic neurons in DAT^{CRE/+}Atg7^{flox/flox} mice. FIG. 1A, Cre immunohistochemistry in DAT^{CRE/+} mouse midbrain. Cre-positive staining (light gray) was seen in the nuclear regions of TH-positive dopamine neurons (light gray) but not in other cell types in the substantia nigra, nor elsewhere in the CNS or in control DAT^{+/+} brain. Bar, 20 μm. FIG. 1B, Representative TH-stained midbrain sections at indicated ages. TH-positive neurons in DAT^{CRE/+}Atg7^{flox/flox} mice (dark staining) progressively decreased in number. Bar, 250 μm. FIG. 1C, Quantification of TH-positive neurons in the substantia nigra pars compacta as in (B). n=3-7 for each group. FIG. 1D, Representative TH-stained (light gray) striatal sections at the indicated age. A progressive decline of mDN axonal projections to the striatum was apparent. Bar, 250 μm. FIG. 1E, Dopamine concentration in striatal tissue was reduced in DAT^{CRE/+}Atg7^{flox/flox} mice at 1- and 6-months of age. n=5-11 per group. **, P<0.01.

FIGS. 2A-2H. Histological characterization of cytoplasmic and dendritic inclusions in DAT^{CRE/+}Atg7^{flox/flox} mice. FIGS. 2A-2E, Double immunostaining of midbrain sections of 1-month and 6-month old DAT^{CRE/+}Atg7^{flox/flox} mice or controls (DAT^{CRE/+}Atg7^{flox/+}). FIG. 2A, anti-ubiquitin and anti-TH stained sections. FIG. 2B, anti-p62/SQSTM1 and anti-TH staining. FIG. 2C, anti-LC3 and anti-TH staining. FIGS. 2D-2E, anti-α-synuclein and anti-TH staining at 6-month (D) and 1-yr (E). n>4 per group. Size bar, 20 μm. FIGS. 2F-2H, Electron microscopic analyses of cytoplasmic/dendritic inclusions. FIG. 2F, Presence of two inclusions (arrows) in cytoplasm on 6-month old midbrain section of DAT^{CRE/+}Atg^{flox/flox} mice. FIGS. 2G-2H, At higher magnification, these inclusions are similar in morphology to Lewy bodies, with both fibrillar and vesicular components and lacking an outer membrane. Inclusions were never observed in control (DAT^{CRE/+}Atg7^{flox/wild}) mice. Bar, 500 nm.

FIGS. 3A-3I. Histological characterization of dopaminergic axonal terminals in DAT^{CRE/+}Atg7^{flox/flox} mice. FIGS. 3A-3C, Double immunostaining of striatal sections from 1-month old DAT^{CRE/+}Atg7^{flox/flox} or control (DAT^{CRE/+}Atg7^{flox/+}) mice. FIG. 3A, anti-VMAT2 and anti-TH staining. FIG. 3B, anti-DAT and anti-TH staining. FIG. 3C, anti-α-synuclein and anti-TH staining. High magnification images of enlarged terminals are provided at right. Similar results were also observed in 6-month old DAT^{CRE/+}Atg7^{flox/flox} mice. n>4 for all groups. Bars, 10 μm. FIGS. 3D-3F, TH-positive dopaminergic axons in striatal sections of 1-month old DAT^{CRE/+}Atg7^{flox/flox} mice were not stained with antibodies for ubiquitin (FIG. 3D), p62/SQSTM1 (FIG. 3E), or LC3 (FIG. 3F). FIGS. 3G-3H, Immunoelectron microscopic ultrastructural analysis of striatal sections from 1-month old control (FIG. 3G; DAT^{CRE/+}Atg7^{flox/+}) or Atg7-deficient (FIG. 3H; DAT^{CRE/+}Atg7^{flox/flox}) mice. Sections were stained with an anti-TH primary antibody and a secondary antibody conjugated to ultra-small gold particles. Many mutant TH-positive terminals (DAT^{CRE/+}Atg7^{flox/flox}) are enlarged relative to normal controls (DAT^{CRE/+}Atg7^{flox/+}). Pink, axon terminal; circle, gold particle. FIG. 3I, Quantification of terminal size. n>500 and 3 mice for each genotype; **, p<0.01.

FIGS. 4A-4G. Atg7 regulates midbrain dopamine neuron survival and axonal morphology through both PI3KI-dependent and independent pathways. FIG. 4A, Atg7-deficient dopaminergic axonal processes from 2-month old DAT^{CRE/+}Atg7^{flox/flox}Pten^{flox/+} mice are increased in size and decreased in density (relative to DAT^{CRE/+}Atg7^{flox/+}Pten^{flox/+} control mice), whereas PTEN-deficient mDNs (from 2-month old DAT^{CRE/+}Atg7^{flox/+}Pten^{flox/flox} mice) display normal appearing dopaminergic axonal processes. Double mutant mice (DAT^{CRE/+}Atg7^{flox/flox}Pten^{flox/flox}) display giant dopaminergic axonal terminals. Arrows indicate TH-positive axonal terminals. Bar, 20 μm. FIG. 4B, Quantification of enlarged axonal terminals as in (A). Terminal size: 4.4-9.8 μm², empty bars; >9.8 μm², black bars. *, p<0.05; **, p<0.01. FIG. 4C, Atg7-deficient mDNs from 2-month old (DAT^{CRE/+}Atg7^{flox/flox}Pten^{flox/+}) mice are reduced in number, whereas PTEN-deficient mDNs from 2-month old (DAT^{CRE/+}Atg7^{flox/+}Pten^{flox/flox}) mice are increased in number. Double mutant DAT^{CRE/+}Atg7^{flox/flox}Pten^{flox/flox} mice display a normal complement of mDNs. Bar, 250 μm. FIG. 4D, Quantification of TH-positive neurons in the substantia nigra as in (C). n=4 for each genotype. FIG. 4E, Increased number of ubiquitin-positive aggregates in TH-positive neurons deficient in PTEN and Atg7 (DAT^{CRE/+}Atg7^{flox/flox}Pten^{flox/flox}) relative to mice deficient in Atg7 alone (DAT^{CRE/+}Atg7^{flox/flox}Pten^{flox/+}). Bar, 10 μm. FIG. 4F, Quantification of ubiquitin-positive aggregates in TH-positive neurons as in (E); n>60 neurons from >4 mice for each genotype. FIG. 4G, Open field behavioral alteration in 4-month old mDN-specific autophagy-deficient mice. Autophagy-deficient (DAT^{CRE/+}Atg7^{flox/flox}Pten^{flox/+}) 4-month old mice display hyperactivity, which is further enhanced in double-mutants deficient in both autophagy and PTEN (DAT^{CRE/+}Atg7^{flox/flox}Pten^{flox/flox}). n>9 in all genotypes; . *, P<0.05; **, P<0.01.

FIGS. 5A-5B. Confirmation of Cre-mediated Atg7 deletion. FIG. 5A, Genomic PCR detection of Cre-mediated excision of loxP-flanked Atg7 gene locus. The deletion of Atg7 gene occurred in midbrain extracts of Dat^{Cre/+}Atg7^{flox/wild} and Dat^{Cre/+}Atg7^{flox/flox} mouse (‘deleted’ band). The excision was not seen in liver extracts, in which the dopamine transporter (DAT) promoter is not active. Atg7 wild-type (wild) bands appeared only in the extracts from Dat^{Cre/+}Atg7^{flox/wild} mouse. FIG. 5B, Decreased levels of Atg7 and LC3-II proteins in midbrain homogenates of Dat^{Cre/+}Atg7^{flox/flox} mouse, analyzed by Western blotting with antibodies as shown. As the midbrain is composed of a complex mixture of cell types in addition to mDNs, ATG7 protein and the presence of LC3-II are only partially reduced; duplicate samples are presented; these data were repeated in 5 sets with consistent results. To address the issue of cell complexity in the homogenates, additional evidence for the specific loss of autophagy in mDNs is provided by immunofluorescent analysis of the accumulation of the autophagy substrate, p62/SQSTM1, only in mDNs (see FIG. 2B).

FIGS. 6A-6C. Progressive loss of TH-positive dopamine neurons in the midbrains of Dat^{Cre/+}Atg7^{flox/flox} mouse. FIG. 6A, Number of TH-positive neurons in substantia nigra pars compacta. Dat^{Cre/+}Atg7^{flox/wild} versus Dat^{Cre/+}Atg7^{flox/flox}; 219.9±7.8 versus 221.8±11.8 (1-mon), p>0.05; 207.2±15.2 versus 123.0±14.6 (3-mon), p<0.01; 241.2±21.0 versus 100.3±21.1 (6-mon), p<0.01 and 223.0±28.0 versus 97.7±2.6 (1-yr), p<0.01. FIG. 6B, Area of TH-positive substantia nigra pars compacta. Dat^{Cre/+}Atg7^{flox/wild} versus Dat^{Cre/+}Atg7^{flox/flox}; 0.357±0.022 mm² versus 0.366±0.022 mm² (1-mon), p>0.05; 0.329±0.026 mm² versus 0.268±0.022 mm² (3-mon), p>0.05; 0.386±0.007 mm² versus 0.234±0.034 mm² (6-mon), p<0.01 and 0.370±0.035 mm² versus 0.245±0.015 mm² (1-yr), p<0.01. FIG. 6C, Cell density of TH-positive neurons per substantia nigra pars compacta. Dat^{Cre/+}Atg7^{flox/wild} versus Dat^{Cre/+}Atg7^{flox/flox}; 627.6±39.9/mm² versus 616.4±32.5/mm² (1-mon), p>0.05; 641.4±57.1/mm² versus 454.2±34.0/mm² (3-mon), p<0.05; 624.1±42.3/mm² versus 423.7±29.5/mm² (6-mon), p<0.01 and 599.2±23.2/mm² versus 401.7±18.1/mm² (1-yr), p<0.01. n=6 (1-mon), 5 (3-mon), 3 (6-mon), and 3 (1-yr).

FIGS. 7A-7D. The contents of dopamine metabolites, serotonin, and serotonin metabolites. FIG. 7A, The striatal content of presynaptic dopamine metabolite, 3,4-dihydroxyphenylacetic acid (DOPAC). Dat^{Cre/+}Atg7^{flox/wild} versus Dat^{Cre/+}Atg7^{flox/flox}; 4.9±0.3 ng/mg versus 6.1±0.5 ng/mg (1-mon), p>0.05 and 6.5±0.8 ng/mg versus 5.0±0.4 ng/mg (6-mon), p>0.05. FIG. 7B, The striatal content of postsynaptic dopamine metabolite, homovanillic acid (HVA). Dat^{Cre/+}Atg7^{flox/wild} versus Dat^{Cre/+}Atg7^{flox/flox}; 8.0±0.5 ng/mg versus 10.5±0.7 ng/mg (1-mon), p>0.05 and 9.7±1.1 ng/mg versus 9.8±0.8 ng/mg (6-mon), p>0.05. FIG. 7C, The striatal content of 5-hydroxytryptamine (5-HT, serotonin). Dat^{Cre/+}Atg7^{flox/wild} versus Dat^{Cre/+}Atg7^{flox/flox}; 3.3±0.2 ng/mg versus 4.4±0.3 ng/mg (1-mon), p<0.05 and 3.5±0.5 ng/mg versus 4.1±0.5 ng/mg (6-mon), p>0.05. FIG. 7D, The striatal content of serotonin metabolite, 5-hydroxyindoleacetic acid (5-HIAA). Dat^{Cre/+}Atg7^{flox/wild} versus Dat^{Cre/+}Atg7^{flox/flox}; 3.0±0.2 ng/mg versus 3.9±0.3 ng/mg (1-mon), p>0.05 and 3.2±0.3 ng/mg versus 3.2±0.4 ng/mg (6-mon), p>0.05.

FIGS. 8A-8B. Immunohistochemical detection of Ubiquitin in the midbrains of 1-mon- and 1-yr-old mice (see FIG. 2A). Ubiquitin immunohistochemistry in the midbrains. FIG. 8A, 1-month-old. FIG. 8B, 6-month-old. Ubiquitin-positive inclusions in 6-month-old Dat^{Cre/+}Atg7^{flox/flox} mice became bigger than those in 1-month-old mouse in TH-positive neurons. Bars, 20 μm.

FIGS. 9A-9B. Immunohistochemical detection of p62/SQSTM1 in the midbrains of 1-month- and 1-yr-old mice (see FIG. 2B). p62/SQSTM1 immunohistochemistry in the midbrains. FIG. 9A, 1-month-old. FIG. 9B, 6-month-old. p62-positive inclusions in 6-month-old Dat^{Cre/+}Atg7^{flox/flox} mice became bigger than those in 1-month-old mouse in TH-positive neurons. Bars, 20 μm.

FIGS. 10A-10B. Immunohistochemical detection of LC3 in the midbrains of 1-month- and 6-month-old mice (see FIG. 2C). FIG. 10A, LC3 immunohistochemistry in the midbrains of 1-month-old mouse. Small LC3-positive inclusions were seen in the cell bodies of TH-positive neurons in Dat^{Cre/+}Atg7^{flox/flox} mouse. Faint and granular staining of LC3 was seen in the cell bodies of TH-positive neurons in Dat^{Cre/+}Atg7^{flox/wild} mouse. FIG. 10B, LC3 immunohistochemistry in the midbrains of 6-month-old mouse. Larger LC3-positive inclusions were seen in the midbrains of Dat^{Cre/+}Atg7^{flox/flox} mice. Most of the LC3-positive inclusions were along the neurites of TH-positive neurons. Bars, 20 μm.

FIGS. 11A-11C. Immunohistochemical detection of VMAT2, DAT, and α-Synuclein in the striatum of 1-month-old mice. FIGS. 11A-11C, VMAT2 immunohistochemistry (FIG. 11A; see FIG. 3A), DAT immunohistochemistry (FIG. 11B; see FIG. 3B), and α-Synuclein immunohistochemistry (FIG. 11C; see FIG. 3C) in the striatum of 1-month-old mouse. These results show that TH-positive axonal axonal processes are VMAT2-positive, DAT-positive, and α-Synuclein-positive. Bars, 10 μm.

FIGS. 12A-12B. Immunohistochemical detection of Ubiquitin and p62/SQSTM1 in the striatum of 1-month-old mice. FIG. 12A, (see FIG. 3D) Ubiquitin immunohistochemistry in the striatum of 1-month-old mouse. No ubiquitin-positive staining was seen in the striatum of Dat^{Cre/+}Atg7^{flox/flox} mouse. This result indicates that TH-positive axonal axonal terminals do not harbor ubiquitin-positive inclusions. FIG. 12B, (see FIG. 3E) p62/SQSTM1 immunohistochemistry in the striatum of 1-month-old mouse. No p62-positive staining was seen in the striatum of Dat^{Cre/+}Atg7^{flox/flox} mouse. This result shows that TH-positive axonal terminals are not p62/SQSTM1-positive inclusions. Bars, 10 μm.

FIGS. 13A-13B. Immunohistochemical detection of LC3 and MAP1 in the striatum of 1-month-old mice. FIG. 13A, LC3 immunohistochemistry in the striatum of 1-month-old mouse. No LC3-positive staining was seen in the striatum of Dat^{Cre/+}Atg7^{flox/flox}. This result indicates that TH-positive axonal axonal processes do not contain LC3-positive inclusions and are therefore different from the midbrain inclusions in Dat^{Cre/+}Atg7^{flox/flox} mouse. See FIG. 3F for details. FIG. 13B, MAP1 immunohistochemistry in the striatum of 1-mon-old mouse. MAP1 staining, a marker for dendrites, was adjacent to TH-positive axonal buttons in the striatum of Dat^{Cre/+}Atg7^{flox/flox} mouse. This result shows that TH-positive axonal terminals contact the dendrites of striatal neurons. Bars, 10 μm.

FIGS. 14A-14C. Atg7 deletion in adult mice causes similar phenotype with Dat^{Cre/+}Atg7^{flox/flox} mouse. FIG. 14A, Experimental procedure diagram of AAV2-Cre/GFP infection into mouse midbrains. See Methods for details. AAV2-Cre/GFP virus was injected into right hemisphere of midbrain of 2-mon-old Atg7^{wild/wild} or Atg7^{flox/flox} mouse (n=3). The mice were sacrificed four or eight weeks after the injection. FIG. 14B, TH-staining of the striatum (upper panels) and midbrain (lower panels) sections from mice sacrificed 4 weeks after the virus injection. Atg7^{flox/flox} mice infected with control AAV2-Cre/GFP did not show any changes in their striatum and midbrain. red, TH; green, GFP. Bars, 30 μm (striatum) and 250 μm (nigra). FIG. 14C, TH-staining of the striatum (upper columns) and midbrain (lower columns) sections from the mice sacrificed 8 weeks after the virus injection. Atg7^{flox/flox} mice infected with AAV2-Cre/GFP had axonal terminal enlargements in their striatum and inclusions in their midbrain, recapitulating the phenotype of the Dat^{Cre/+}Atg7^{flox/flox} mice. Neither Atg7^{flox/wild} mice infected with AAV2-Cre/GFP nor Atg7^{flox/flox} mice infected with AAV2-GFP showed changes in their striatum and midbrain. These results confirm that the phenotypes in Dat^{Cre/+}Atg7^{flox/flox} mouse are not developmental defects. red, TH; green, GFP. Bars, 30 μm (striatum), 250 μm (nigra) and 100 μm (nigra).

FIG. 15. Additional examples of immunoelectron microscopy using TH-specific antibodies in the striatum of Dat^{Cre/+}Atg7^{flox/flox} mice or controls; see FIGS. 3G-3I for details.

FIGS. 16A-16D. Primary midbrain neuronal cultures from autophagy-deficient Dat^{Cre/+}Atg7^{flox/flox} mice display prolonged neurites. FIG. 16A, Elongated processes in TH-positive neurons in primary midbrain cultures from autophagy-deficient (Dat^{Cre/+}Atg7^{flox/flox}) mice relative to controls (Dat^{Cre/+}Atg7^{flox/wild}). FIG. 16B, The elongated process phenotype is also apparent in primary cortical cultures from (Atg7^{flox/flox}) mice transfected with Cre-recombinase encoding plasmid (along with GFP plasmid) compared to primary neurons transfected with GFP plasmid alone. The application of two different PI3K inhibitors, 10 μM Wortmannin or 50 μM LY294002, reversed the process length phenotype. FIG. 16C, Quantitation of process length as in (A). Dat^{Cre/+}Atg7^{flox/wild} versus Dat^{Cre/+}Atg7^{flox/flox}; 0.933±0.098 mm versus 1.704±0.139 mm, p<0.01. n>49 neurons per group in at least three separate independent experiments. FIG. 16D, quantification of data as in (C). Dat^{Cre/+}Atg7^{flox/wild} versus Dat^{Cre/+}Atg7^{flox/flox}; 0.428±0.036 mm versus 0.721±0.073 mm (DMSO), 0.198±0.018 mm versus 0.217±0.021 mm (Wortmannin, WM), and 0.158±0.015 mm versus 0.176±0.018 mm (LY294002, LY). n>45 neurons per group in at least three separate independent experiments. Bar, 50 μm.**, P<0.01.

FIGS. 17A-17B. Loss of dopamine neurons in Dat^{Cre/+}Atg7^{flox/flox} mice was rescued by PTEN deficiency. FIG. 17A, Numbers of TH-positive neurons in substantia nigra pars compacta. 199.5±11.7 (Dat^{Cre/+}Atg7^{flox/wild} Pten^{flox/wild}) 152.5±11.6 (Dat^{Cre/+}Atg7^{flox/flox} Pten^{flox/wild}), 247.9±16.4 (Dat^{Cre/+}Atg7^{flox/wild}Pten^{flox/flox}), and 207.3±11.1 (Dat^{Cre/+}Atg7^{flox/flox}Pten^{flox/flox}). FIG. 17B, Area of TH-positive neurons in substantia nigra pars compacta.

FIG. 18. Model of autophagy regulation of axonal terminal morphology and motor behavior. Autophagy appears to normally function to suppress axonal terminal size, at least in part through the suppression of PI3KI-mediated regulation. PI3KI functions to regulate axonal terminal size downstream of autophagy. Finally, prior studies in other cellular systems indicate that the PI3K pathway can negatively regulate autophagy.

FIGS. 19A-19D. FIG. 19A, Mutant LRRK2 pathology is suppressed by Mitotracker dye analysis of primary neuronal cultures that overexpress either wild-type or G2019S mutant LRRK2; FIG. 19B, Mutant LRRK2 appears to interact with the AKT signaling pathway. Overexpression of a constitutively active form of AKT1 (c.a.-AKT1) dramatically increases process length and branching, particularly with respect to the longest process. Co-expression of G2019S mutant, but not WT, LRRK2 completely suppresses this phenotype. N=10 per group. *, P<0.05. FIG. 19C, c.a.-AKT1 fails to rescue the inclusion phenotype of the G2019S expressing neurons, and thus this phenotype is separable from survival. Arrows point to inclusions. FIG. 19D, Constitutively active (c.a.) AKT1 or dominant negative (d.n.) GSK3β rescue the decreased neuronal survival associated with G2019S expressing neurons. Similarly, these constructs rescue the neurite length phenotype, but not the inclusion formation, found in G2019S expressing cells. N=10 for each group.

FIG. 20. PTEN RNAi (genetic suppression) prevents the neurite loss phenotype observed with LRRK2 G2019S mutant overexpression in primary neurons (a PD cell model), and mimics the phenotype of LRRK2 RNAi.

FIG. 21. Model of autophagy regulation.

FIGS. 22A-22B. FIG. 22A, Representative TH-stained striatal sections at 1-month of age. Large TH-positive buttons and progressive decline of axonal projections were seen. FIG. 22B, Electron microscopic analyses of striatal axonal buttons. Enlarged synaptic terminals (arrows) but otherwise normal morphology in 1-month old Dat^{Cre/+}Atg7^{flox/flox} sections relative to controls.

FIG. 23. Representatives of TH-stained striatal sections at indicated genotype in 2-month old animals. Much larger TH-positive buttons were seen in Dat^{Cre/+}Atg7^{flox/flox}Pten^{flox/flox} mice. No TH-positive buttons were seen in Dat^{Cre/+}Pten^{flox/flox} mice.

### DETAILED DESCRIPTION

Components of a PTEN cell signaling pathway may be utilized as therapeutic targets for treating, preventing, slowing the progression of or delaying the onset of a neurodegenerative disease or disorder. Such components that may be targeted include, but are not limited to, phosphatase and tensin homologue (PTEN), glycogen synthase kinase 3 beta (GSK3β), and AKT (also referred to as protein kinase B (PKB)). The subject matter disclosed herein relates to the therapeutic use of compounds that activate a phosphoinositide-3 kinase (PI3K) pathway, for example, inhibitors of PTEN, inhibitors of GSK3β, or activators of AKT.

The PTEN/PI3K/GSK3β/AKT intracellular signaling pathway functions to transmit signals within a cell that regulate cell growth, proliferation and survival, and other cellular processes. PTEN is an enzyme (specifically, a phosphatase) that acts as part of the pathway to signal cells to stop dividing and may trigger cells to undergo a form of programmed cell death called apoptosis. PTEN functions to antagonize or counter the activity of another signaling pathway component, PI3K. When PTEN is active, it dephosphorylates phosphatidylinositol (3,4,5)-trisphosphate (PIP3), yielding phosphatidylinositol (4,5)-bisphosphate (PIP2). When PI3K is active, it phosphorylates PIP2 back to PIP3. AKT, a signaling pathway component located downstream of PI3K, becomes active when it is bound by PIP3. GSK3β is a signaling pathway component located downstream of AKT; when AKT is active, GSK3β is inhibited. Thus, active PI3K or active AKT (or both) promote cell growth and survival, while inactive PTEN or inactive GSK3β (or both) promote cell growth and survival.

A method is provided for treating a neurodegenerative disease or condition in a subject, the method comprising administering to the subject an effective amount of a PTEN inhibitor, a GSK3β inhibitor or an activator AKT.

In some embodiments, the compound may be, for example, a small organic molecule, a nucleic acid (for example, DNA, RNA, DNA/RNA, small interfering RNA (siRNA), antisense RNA, short hairpin RNA (shRNA), double stranded RNA (dsRNA) or cDNA), a peptide, a protein, a peptidomimetic or an antibody.

In some embodiments, the neurodegenerative disease or condition may be Parkinson's disease, Alzheimer's disease, Lewy body dementia, Creutzfeldt-Jakob disease, Huntington's disease, multiple sclerosis or amyotrophic lateral sclerosis (ALS).

In some embodiments the compound can be administered directly to a site of therapeutic interest in a subject, for example, an organ, tissue or cell of the subject, for example, brain, spinal cord or neurons, including motor neurons or dopamine neurons. In other embodiments, the compound comprises a carrier or signal which directs the compound to an organ, tissue or cell of the subject.

The effective amount of the disclosed compounds can vary depending on the condition, severity of the symptoms presented and the particular subject being treated. One of skill in the art would readily be able to determine the amount of compound required. Compounds may be administered in combination with one or more additional compounds known to be useful in the treatment or prevention of neurodegenerative diseases or disorders, or the symptoms thereof.

PTEN Inhibitors

Experimental results described in Example 1 show that genetic suppression of phosphatase and tensin homologue (PTEN) in an autophagy-deficiency mouse model of neurodegeneration reduces the loss of dopamine neurons in the brain and increases

Examples of PTEN inhibitors that may be used in some embodiments include small molecules that inhibit or suppress PTEN expression or activity, or structural or functional analogs of such small molecules.

Vanadium complexes comprising, for example, vanadate (VO) or bisperoxovanadate (bpV) complexed to one or more organic ligands, may be used to inhibit PTEN in some embodiments. Such complexes comprise a VO or bpV complexed with a ligand. Non-limiting examples of ligands include 1-isoquinoline (isoqu), phenanthroline (phen), phenylbiguanide (biguan), 3-hydropicolinate (OH-pic), bipyridine (bipy) and picolinato (pic). See examples of such small molecules disclosed in Rosivatz E, et al. “A small molecule inhibitor for phosphatase and tensin homologue deleted on chromosome 10 (PTEN)”, ACS Chem. Biol. 2006 Dec. 15; 1(12):780-90; which is herein incorporated by reference. PTEN inhibitors including bpV(bipy), bpV(OHpic), bpV(phen), bpV(pic) are available from commercial vendors.

Compounds described in U.S. Patent Application Publication No. 2007/0203098 (“PTEN Inhibitors”) may also be used to inhibit PTEN in some embodiments.

PTEN may also be inhibited by compounds that suppress transcription or translation of a gene encoding PTEN. For example, nucleic acids such as siRNA, antisense RNA, or shRNA. Based on known nucleic acid sequence information for PTEN (see, for example, GenBank Accession No. NM_{—}000314), one of ordinary skill in the art would understand how to suppress or inhibit expression of a gene or nucleic acid encoding PTEN.

PTEN activity may also be inhibited by an antibody, or fragment thereof, that specifically binds to PTEN. Using the nucleic acid and amino acid sequence information known for PTEN, one of ordinary skill in the art would understand how to make and use an antibody that specifically binds PTEN.

GSK3β Inhibitors

Inhibition of glycogen synthase kinase 3 beta (GSK3β) blocks neuronal toxicity of a PD-associated LRRK2 mutant (see Example 2). Therefore, a compound used in the disclosed methods may be an inhibitor of GSK3β. Such inhibitor may suppress or inhibit GSK3β activity or expression. The inhibitor may be small molecules, or structural or functional analogs of such small molecules. Classes of GSK3β inhibitors that can be used in the disclosed methods include, but are not limited to, thiazole, bis-indole, aminopyrimidine, arylindolemaleimide, and benzazepinone. Examples of small molecule inhibitors include SB216763, AR-A014418, CHIR98014, indirubin-3′-monoxime, alsterpaullone and kenpaullone. A PTEN inhibitor may also be an ion, for example, lithium chloride.

Non-limiting examples of small molecule GSK3β inhibitors include compounds described in Selenica M-L, et al. Efficacy of small-molecule glycogen synthase kinase-3 inhibitors in the postnatal rat model of tau hyperphosphorylation. Br J Pharmacol (2007) 152: 959-979, which is herein incorporated by reference.

Additional examples of GSK3β inhibitors include compounds that target (for example, inhibit or activate) a pathway component upstream of GSK3β, causing or resulting in subsequent inhibition of GSK3β.

GSK3β may also be inhibited by compounds that suppress transcription or translation of a gene encoding GSK3β. For example, nucleic acids such as siRNA, antisense RNA, or shRNA. Based on known nucleic acid sequence information for GSK3β (see, for example, NM_{—}002093), one of ordinary skill in the art would understand how to suppress or inhibit expression of a gene or nucleic acid encoding GSK3β.

GSK3β activity may also be inhibited by an antibody, or fragment thereof, that specifically binds to GSK3β. Using the nucleic acid and amino acid sequence information known for GSK3β, one of ordinary skill in the art would understand how to make and use an antibody that specifically binds GSK3β.

AKT Activators

AKT (or protein kinase B) is a downstream component of a PTEN/PI3K/GSK3β/AKT pathway that regulates cell growth and proliferation. AKT activation inhibits neuronal toxicity of a PD-associated LRRK2 mutant (see Example 2). LRRK2 and AKT both lead to altered process length and the invention provides that the two ‘co-suppress’ one another. This shows that they may converge on the same point in a signal transduction pathway (or each function at multiple points along a pathway). LRRK2 and AKT both ultimately regulate survival in primary neurons. Glutamate excitotoxicity is involved in the phenotype of LRRK2 mutant cells, and the AKT/GSK3β pathway impinges on LRRK2 toxicity and can suppress it. Thus, a compound used in the described methods activates an AKT (protein kinase B) protein.

An activator of AKT may be, for example, a compound that increases the expression or activity of AKT, or a compound that targets (for example, inhibits or activates) a pathway component upstream of AKT, causing or resulting in subsequent activation of AKT.

Activators of AKT may include nucleic acid sequences (for example, DNA or RNA) or amino acid sequences (for example, peptides or polypeptides) that increase or activate AKT expression or activity. For example, overexpression of AKT from a plasmid. Based on known nucleic acid sequence information for AKT (see, for example, GenBank Accession No. NM_{—}005163), one of ordinary skill in the art would understand how to suppress or inhibit expression of a gene or nucleic acid encoding AKT.

Autophagy and Parkinson's disease

Autophagy. Macroautophagy (herein termed autophagy) is a self-preservation mechanism by which long-lived cellular proteins, bulk cytoplasmic constituents, and whole organelles are delivered to lysosomes for degradation. The autophagy degradation pathway is thought to complement the ubiquitin-proteasome degradation pathway, which primarily handles short-lived proteins. The ultrastructural hallmark of macroautophagy is the presence of double membrane vesicles and multivesicular bodies which arise from an unknown cellular compartment, encircle cargo, and then fuse with lysosomes compartment. Autophagy was initially observed in the context of starvation in the mammalian liver, and was presumed thereby to be a protective cellular response that allows for recycling of limited nutrients. Autophagy is also induced in the context of toxins, such as a number of chemotherapeutic agents. Additionally, autophagy is observed developmentally in the context of major morphological changes, such as metamorphosis in Drosophila. This underscores the high degree of conservation of the autophagy degradation pathway, which is observed in eukaryotes from yeast to man.

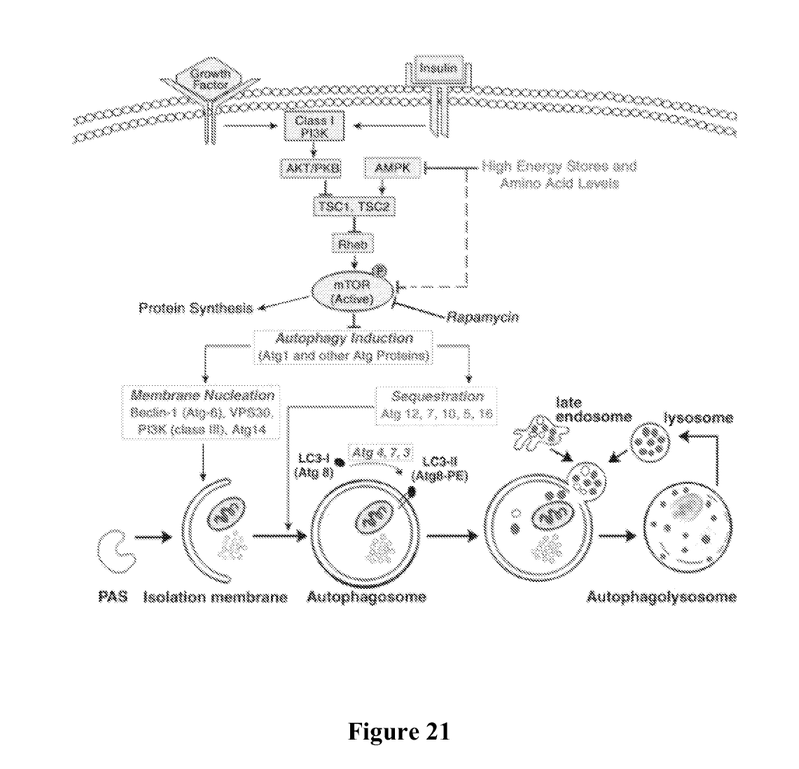

Autophagy Genetics. In yeast, autophagy is essential for survival under hostile conditions, such as nutrient deficiency. This observation has allowed for the identification of a number of genes that are required for autophagy, and autophagy genes have been organized within a tentative pathway vis-á-vis the autophagy process (FIG. 21). Several genes encode proteins within an autophagy induction complex, including the serine/threonine kinase ATG1 (for autophagy-1). Additional genes direct cargo to the autophagy machinery, and these appear to function in a cargo-specific manner. Next, vesicle nucleation is directed by a set of genes that include the type 3 phosphatidylinositol 3-kinase (PI3K) VPS34 and ATG6 (mammalian Beclin). Finally, vesicle maturation requires two sets of ubiquitin-like proteins: ATG12, a ubiquitin-like protein, is covalently attached to ATG5; and ATG8 (mammalian LC3), a ubiquitin-like protein, is modified by the lipid phophatidylethanolamine. Both of these processes require ATG7. Additional genes are implicated in the retrieval and breakdown of vesicles. There is surprising evolutionary conservation of the autophagy machinery from yeast to man. Thus, ATG1, ATG5 ATG7, ATG8 (mammalian LC3), and other yeast molecules in the pathway have identified

mammalian orthologues. Knockout mice deficient in ATG5 or ATG7 show a specific deficit in autophagy, and die within 24 hours of birth. Furthermore, the modification of ATG5 by ATG8/LC3 is conserved, and mammalian LC3 localizes to autophagosomes.

Regulation of Autophagy. Mechanisms of autophagy regulation and induction are highly conserved. Thus, the autophagy initiator complex, including ATG1, appears to be highly regulated by nutrient status through the type I PI3K/AKT/mTOR (for Target of Rapamycin) pathway (FIG. 21). Type I PI3K activity converts phosphatidylinositol (PtdIns)(4,5)P2 (herein termed PIP2) to PtdIns (3,4,5)P3 (herein termed PIP3). This is counterbalanced by the lipid phosphatase PTEN, which reduces PIP3 accumulation. PIP3 induces AKT and, in turn, lead to activation of TOR kinase, and ultimately to inhibition of autophagy. The precise mechanism by which mTOR activity inhibits autophagy is not clear. AKT kinase activity, both directly and indirectly (through the tuberous sclerosis complex proteins 1 and 2 [TSC1 and TSC2]), leads to mTOR phosphorylation. The PI3K/AKT/mTOR pathway is induced upstream primarily through growth factor and insulin receptor stimulation. mTOR is also negatively regulated by high energy stores through another kinase pathway, the AMPK pathway, which senses the availability of specific nutrients such as amino acids and ATP levels. An additional mechanism of autophagy regulation is through type III PI3Ks(which convert PI to PIP1), and include Vps34, which complexes with ATG6/Beclin to induce autophagy.

Basal Autophagy in CNS Neurons. Unlike liver cells, neurons in the CNS do not typically display autophagy induction in the context of starvation of the whole organism, because of the tight control placed on nutrient availability to the brain. Furthermore, autophagy induction in the liver allows for an increase in nutrient supply to the brain. Nonetheless, there is a low level of constitutive autophagy that is seen in neurons, particularly at distal neurite processes. This basal level of autophagy may play a role in the structural plasticity of neurites, as well as in mechanisms of functional plasticity such as long-term potentiation of synaptic transmission. Also, autophagy may be required for degradation of misfolded or damaged proteins in long-lived postmitotic neurons. As macroautophagy is the only mechanism by which whole organelles are degraded, it is clear that, in the lifetime of a postmitotic CNS neuron, autophagy most likely plays a critical housekeeping role.

Direct evidence for the role of autophagy in neuron maintenance came from studies of mice lacking either ATG7 or ATG5 only in CNS neurons, using a CRE-LoxP approach. Briefly, Nestin-CRE mice, which harbor the bacterial CRE recombinase under the control of a pan-neuronal Nestin promoter, were crossed to animals that have either ATG5 or ATG7 genomic sequences flanked by LoxP target sites for the CRE recombination. After back-crossing, mice were generated that are homozygous for the LoxP flanked genes and express brain neuron-specific CRE, and thus delete an essential autophagy element specifically in CNS neurons. By postnatal day 14 (P14), these mice display neuronal behavioral deficits, and by two months of age these mice show diffuse CNS neuronal loss and ubiquitin-positive inclusions. The mDN phenotype has not been directly explored in these animals. Thus, autophagy appears to play an essential basal role in neurons. The limited observation of autophagic vacuoles in wild-type CNS neurons in the absence of stressors implies that autophagic vesicles must turn over rapidly in the CNS. The precise role of basal autophagy remains an open question. In a recent study, mice were generated with a specific deletion of ATG7 in cerebellal Purkinje cells. These mice initially showed axonal dystrophy and disrupted axonal architecture. This contrasts with the observation of axonal terminal enlargement with a normal appearance (see below in Examples 1 and 3). However it is possible that this phenotype is partially a consequence of developmental abnormalities, as ATG7 was deleted relatively early, and as cerebellar development proceeds quite late. Furthermore, this study did not specifically probe the mechanism of these the autophagy mediated phenotypes, or the role of autophagy in disease. The studies in Examples 1 and 3 show that mDNs may behave differentially, suggesting cell type specific roles for autophagy in neuron classes, and presenting a rationale for why an alteration in a general cellular process such as autophagy may lead to the preferential loss of a single neuron class in a human disease.

Environmental Stressors and Autophagy in Neurons. In the context of stressful insults such as toxin exposure or axotomy, neurons display robust autophagy induction, with accumulation of autophagosomes. This observation has most commonly been interpreted as a cellular response to stressors in the context of remodeling. Alternatively, autophagy induction may reflect a non-apoptotic form of cell death, also termed type 2 programmed cell death (PCD; in contrast with type 1 apoptotic death). Type 2 PCD is most commonly seen in developmental processes, such as the massive cell death observed in the course of insect metamorphosis. This form of autophagy is believed to be a nutrient-conserving form of death that is of benefit to the organism as a whole. In the context of environmental stressors in neurons, it is thus unclear whether autophagy induction is an active mechanism of suicide or a cellular response to injury.

In one study, the role of autophagy in the context of a dopaminergic toxin, 1-methyl-4-phenylpyridinium-(MPP+) induced cell death, implicated in Parkinsonism. Dopaminergic cell death in the context of this toxin is mediated by autophagy activation through induction of mitogen-activated protein kinase/extracellular signal-regulated protein kinase kinase (ERK). These data underscore the important role of autophagy in mDN survival, and the need to define precisely the mechanism of action by which toxic or genetic stressors induce mDN alterations in the context of the intact mammalian brain.

Autophagy and Neurodegeneration. The accumulation of autophagosomes has long been noted in the context of Parkinson's disease and other neurodegenerative disorders. Autophagy induction has generally been viewed as a protective or adaptive cellular response to damaged proteins and organelles in this context, but autophagy may also represent an active form of cell death. Thus a basic question is whether autophagic is good or bad in neurodegeneration.

Autophagy and Parkinson's Disease. PD is characterized by the progressive loss of mDN, and is the second most common neurodegenerative syndrome. Importantly, other neurons are also affected in PD in the CNS and elsewhere, consistent with a broad cellular defect. A hallmark of PD is the presence of Lewy body intracellular cytoplasmic proteinaceous inclusions, and these consist in part of α-Synuclein (αSyn) and ubiquitin protein aggregates. The presence of intraneuronal protein aggregates in the disease process suggest the possibility of a defect in protein degradation leading to PD. This is further supported by reports of autophagy induction as well as proteasome defects in the context of PD. However, whether the aggregates are causal, represent a protective cellular response, or are simply epiphenomenon is not clear. Of note, both αSyn and ubiquitin are also implicated in PD based on genetic analysis of rare familial cases of Parkinsonism. Mutations in αSyn—both gene multiplications leading to increased protein levels and missense mutations that may increase the propensity of αSyn to aggregate—are associated with autosomal dominant Parkinsonism. αSyn may be degraded in part through the autophagy pathway, and through a related pathway termed chaperone-mediated autophagy which utilizes a specific receptor on lysosomes, LAMP1, for direct uptake of substrates. Mutations in Parkin lead to an autosomal recessive form of Parkinsonism, and there is evidence that Parkin is an E3 ubiquitin ligase involved in specifying substrates for ubiquitin modification. Thus, analysis of genetic forms of Parkinsonism support the notion that defective protein degradation may be involved in PD.

Mutations in LRRK2 were described as the most common autosomal dominantly inherited form of Parkinson's disease, and the most common mutations appear to lead to activation of kinase activity. The role of LRRK2 activation has been investigated in the context of primary neurons, and results show that LRRK2 activation leads to prominent activation of the autophagy/lysosomal pathway and regulates neurite process morphology. See International Patent Application Publication No. WO 2007/124096.

The PI3K/PTEN/AKT Pathway, PD, and Autophagy. An additional link between autophagy regulation and PD comes from data that associate the PI3K/PTEN/AKT pathway with familial forms of Parkinsonism. As described above, autophagy is regulated in part by type I PI3K leading to the accumulation of PIP2. This is counterbalanced by the lipid phosphatase PTEN, which reduces PIP2 accumulation. PIP2 induces AKT activation and, in turn, activation of TOR kinase, leading ultimately to inhibition of autophagy. The PI3K/PTEN/AKT pathway has also been linked to familial forms of Parkinsonism. DJ-1, a gene that is mutated in autosomal recessive form of familial Parkinsonism, is reported to function normally to repress PTEN activity. Thus, a pathological mechanism for DJ-1 mutation in familial Parkinsonism may be increased PTEN activity and induction of autophagy. A third gene linked to autosomal recessive Parkinsonism is termed PTEN-induced kinase 1, or Pink1. Pink1 expression appears to be dependent on PTEN activity. Taken together, these data potentially link familial forms of Parkinsonism with a major regulatory pathway for autophagy. However, given the pleotropic activity of the PI3K/PTEN/AKT pathway, additional data are needed to establish any link.

Animal Models of PD. A challenge in the context of Parkinson's disease (PD) research has been the generation of animal models that correctly recapitulate the disease state. Surprisingly, animals that harbor genetic mutations mimicking familial forms of Parkinsonism, including αSyn, Parkin, DJ-1, and Pink1 mutations, fail to show clear evidence of the progressive mDN loss and proteinateus intracytoplasmic neuronal inclusions, termed Lewy Bodies, that typify the disease state. Another approach that has been taken to generate PD disease models is the use of oxidative toxins, such as MPTP, 6-OHDA, rotenone, or others. All of these lead to a variable degree of dopamine neuron toxicity, but other features of PD pathology, such as Lewy body aggregates, are not prominent in these models. Finally, proteasome inhibitors applied systemically have been reported to lead to PD-like pathological features in rodents, but these studies have been difficult to replicate. The lack of PD pathology in toxin and genetic animal models may be a result of differences in protein degradation between aging mice and humans. This may relate to the more challenging condition of human mDNs, which are substantially longer lived and considerably more structurally complex. Alternatively, other species differences may exist in terms of the efficacy of protein degradation mechanisms. Examples 1 and 3 focuses on the role of autophagy in the context of dopamine neuron survival, and relate this to genetic and toxin models of the disease.

Autophagy as a Double-Edged Sword. A number of studies show a causal role for autophagy in both cell survival and cell death outside of the CNS. In interleukin-3 (IL-3) dependent bone marrow-derived cells that cannot undergo apoptosis due to deletion of 2 genes (Bax and Bak), cells nonetheless die during growth factor depletion. In the absence of autophagy, the cell death is greatly accelerated. Thus, autophagy allows for continued survival in the absence of nutrients or growth factors. In contrast, a second set of experiments support an active role for autophagy in cell death. For instance, caspase-inhibitor induced autophagic cell death is dependent on the activity of autophagy genes including ATG7 and Beclin, as knocking down either of these prevents cell death. In dopaminergic neurons, there is evidence that autophagy plays a causal role in toxin induced cell death, and that autophagy is induced in the context of neurodegeneration syndromes, such as Alzheimer's and Parkinson's diseases.

Terms

In some embodiments, the compound can be combined with a carrier. The term “carrier” is used herein to refer to a pharmaceutically acceptable vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “effective” is used herein to indicate that the inhibitor is administered in an amount and at an interval that results in the desired treatment or improvement in the disorder or condition being treated (e.g., an amount effective to arrest, delay or reverse the progression of prostate cancer).

In some embodiments, nonlimiting examples of the subject include: human, mouse, rabbit, monkey, rat, bovine, pig or dog.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

The following examples are illustrative, and are set forth to aid in the understanding of the disclosed embodiments, and should not be construed to limit in any way the scope of the invention as defined in the claims which follow thereafter.

### EXAMPLES

### Example 1

### Genetic Inhibition of PTEN Suppresses Loss of Dopamine Neurons in an Autophagy-Deficient Mouse Model

Parkinson's disease (PD) is a chronic and progressive neurodegenerative disorder characterized by motor impairments including slowed movements, gait difficulty, and a tremor at rest¹. PD pathological findings including midbrain dopaminergic neurons (mDN) loss and the presence of cytoplasmic inclusions termed Lewy bodies composed of ubiquitin, α-Synuclein, and other components. Additionally, macroautophagy (‘autophagy’ herein) alterations are apparent in mDNs of PD patients, which likely reflect defects in the autophagy-lysosomal degradation pathway^{2,3}. Autophagy is an evolutionarily conserved mechanism for bulk intracellular degradation of proteins and organelles. Pathological studies have implicated defective autophagy in neurodegenerative disorders, including PD. Autophagy is induced in the context of starvation and other stressors, and defective autophagy reduces neuron viability^{4,5}. Genetically altered mice that are deficient in autophagy within specific neuronal populations in the cerebral cortex, hippocampus, and cerebellum display reduced neuron survival and harbor ubiquitin-positive inclusions in soma. Furthermore axonal processes appear dystrophic and swollen with inclusions⁵⁻⁷.

This Example investigates the mechanism of inclusion formation in mDNs, the relationship of these inclusions to mDN demise, and the role of the PD-associated gene α-Synuclein. Furthermore, the studies in this Example probe the physiological function of an essential autophagy component, Atg7, in mDN morphology and mDN-associated motor behavior. Results show that, at a molecular level, these Atg7-associated functions are mediated through the phosphoinositol-3-kinase class I signaling pathway (PI3KI) pathway. Finally, PTEN deficiency rescues the mDN loss associated with Atg7 deficiency.

Progressive Loss of Atg7-Deficient mDNs

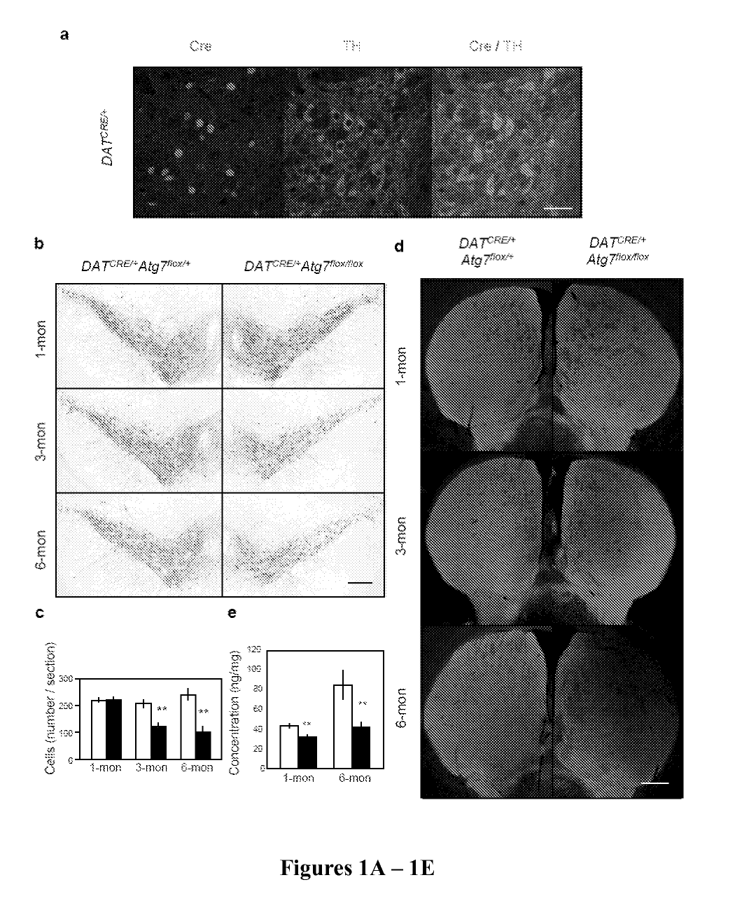

Genetically altered mice were generated in which an essential component of the autophagy pathway, Atg7⁸, is deficient specifically in mDNs. mDN-specific Atg7-deficient animals were generated by crossing mice that express CRE recombinase under the control of the endogenous promoter of a postmitotic mDN-specific gene, the dopamine transporter⁹ (DAT^{CRE/+} knock-in mice) with Atg7^{flox/flox} mice¹⁰. Expression of Cre recombinase was specific to tyrosine hydroxylase (TH; the rate limiting enzyme of dopamine biosynthesis) positive midbrain neurons (FIGS. 1A and 5A), and excision of loxP sequence-flanked Atg7 by the transgene-encoded CRE recombinase was detected in midbrain genomic DNA of mutant mice. Level of Atg7 protein was reduced in midbrain homogenates from DAT^{CRE/+}Atg7^{flox/flox} relative to control mice (DAT^{CRE/+}Atg7^{flox/+} or DAT^{CRE/+}Atg7^{+/+}), consistent with defective autophagic vacuole formation (but this was diluted by the presence of intact Atg7 in all other midbrain cell types; FIG. 5B). Taken together, these data confirm the specific loss of autophagy within mDNs in the mutant mice.

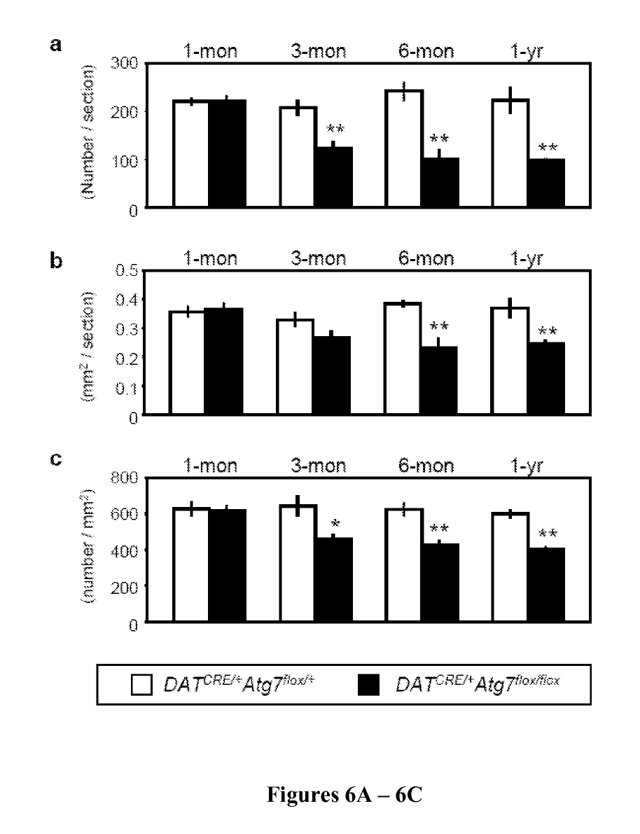

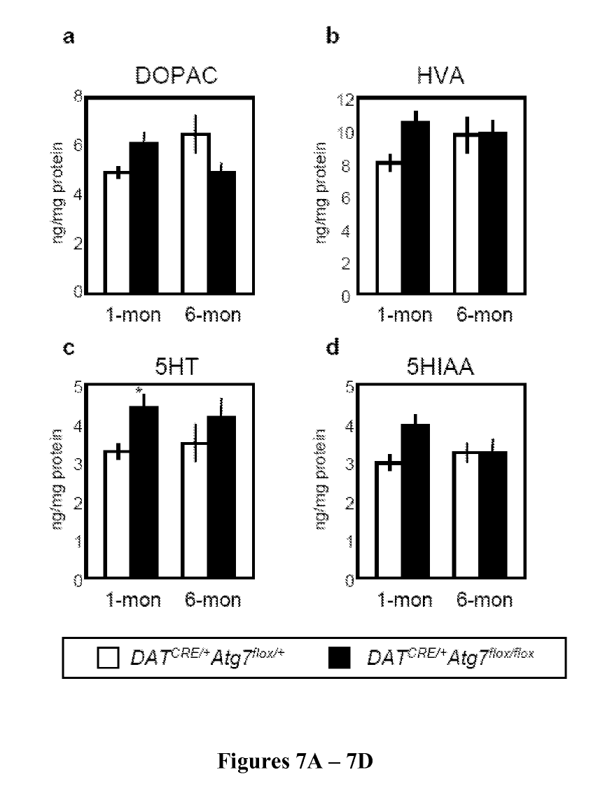

DAT^{CRE/+}Atg7^{flox/flox} mice displayed a normal complement and density of TH-positive mDNs at 1-month of age (FIGS. 1B-1C and 6). The number and density of TH neurons, however, declined progressively at 3- and 6-month time points, and by 1 year over 70% of the TH-positive cells were lost relative to control animals (FIGS. 1B-1C and 6). Substantia nigra mDN axons project to the striatum, and DAT^{CRE/+}Atg7^{flox/flox} mice showed a progressive reduction in dopaminergic axonal density within the striatum, as expected (FIGS. 1D and 6B-6C). Striatal dopamine content was decreased by 27% at 1 month of age, and the reduction became more severe at 6 months (approximately 50%; FIGS. 1E and 7). Thus, DAT^{CRE/+}Atg7^{flox/flox} mice exhibited a progressive loss of midbrain dopamine neurons and a reduction of dopaminergic axonal density and dopamine content in the striatum.

### α-Synuclein-Positive Inclusions in Aged Atg7-Deficient Dopamine Neurons

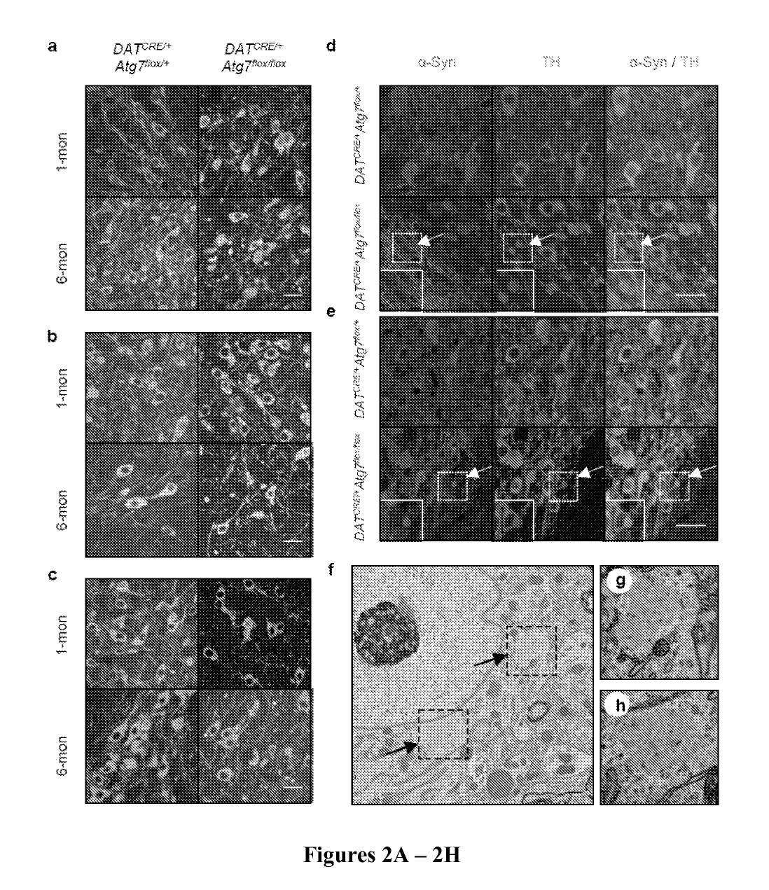

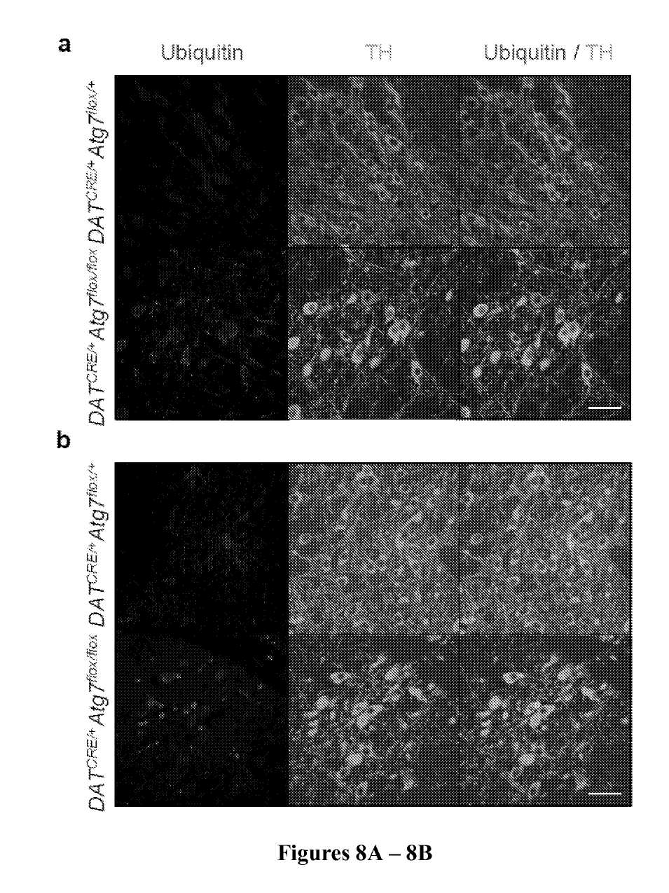

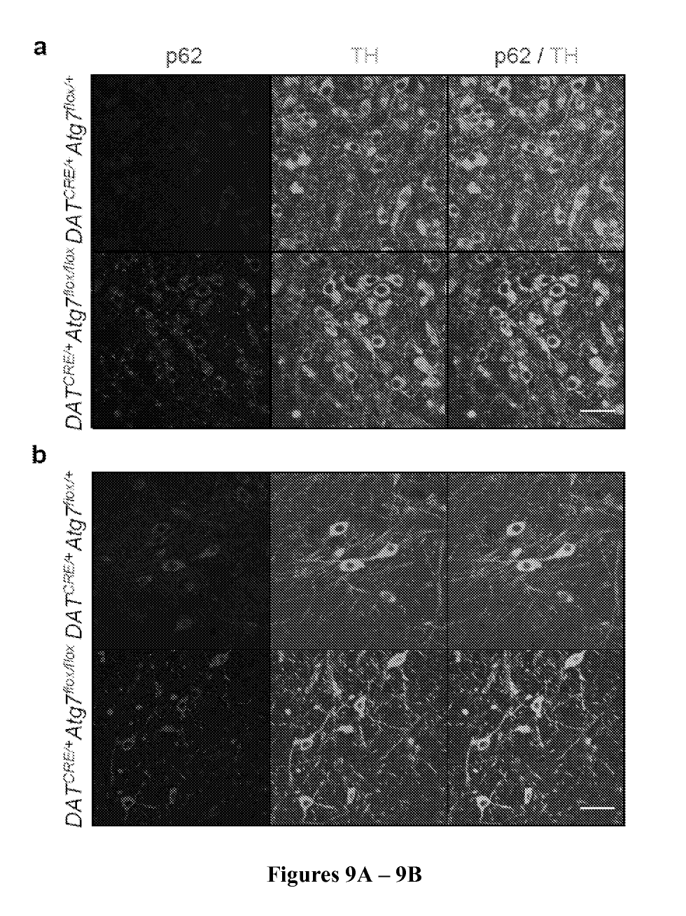

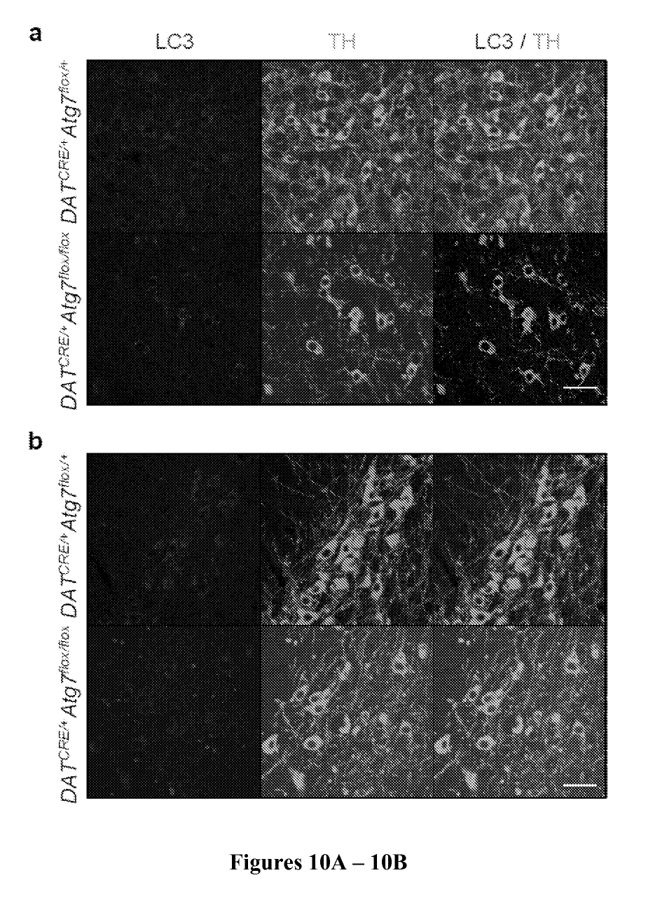

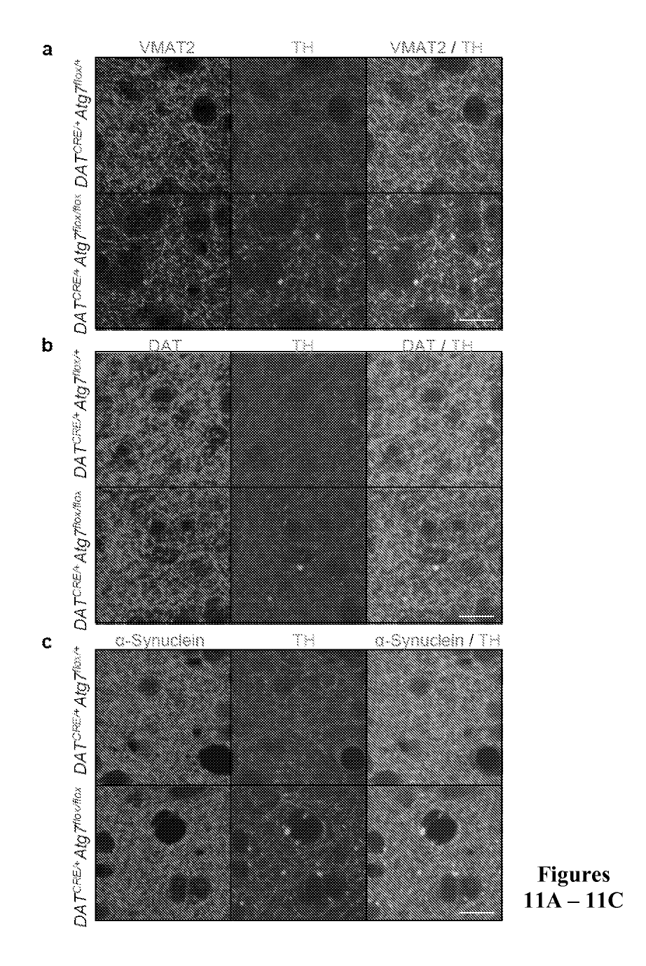

Numerous ubiquitin-positive inclusions were apparent in Atg7 mutant mDN TH-positive cell bodies and dendrites from 1-month of age, whereas these were never seen in controls (FIGS. 2A and 8). These inclusions stained with antibodies to p62/SQSTM1 and LC3″, components of the autophagy machinery pathway (FIGS. 2B-2C, 9 and 10). Of note, the morphology and localization of inclusions progressed over time. In 1-month old mutants, aggregates were relatively small and localized to the cell body of TH-positive neurons, whereas at later time points—up to 1-year old mutants—inclusions were greatly enlarged and extended into dendrites. Inclusions stained positively for α-Synuclein in mutant mDNs of 1-year old mice but not at 6-months or earlier time points (FIGS. 2D-2E). Electron microscopic examination revealed that inclusions in autophagy-deficient mDNs are composed of both filamentous and vesicular elements, akin to the ultrastructure of Lewy body inclusions in PD¹² (FIGS. 2F-2H). As recruitment of α-Synuclein to inclusions occurs only at a late time point in the autophagy-deficient mDNs, it is not likely that α-Synuclein nucleation and aggregation are primary events in inclusion formation. Consistent with this, transgenic overexpression of a clinical mutant form of α-Synuclein with a propensity to aggregate, A53T¹³, in the context of autophagy deficient DAT^{CRE/+}Atg7^{flox/flox} mutant mice, did not modify inclusion formation or neuron survival at up to 1 year of age.

### Atg7-Mediated Regulation of Dopaminergic Axonal Terminal Morphology

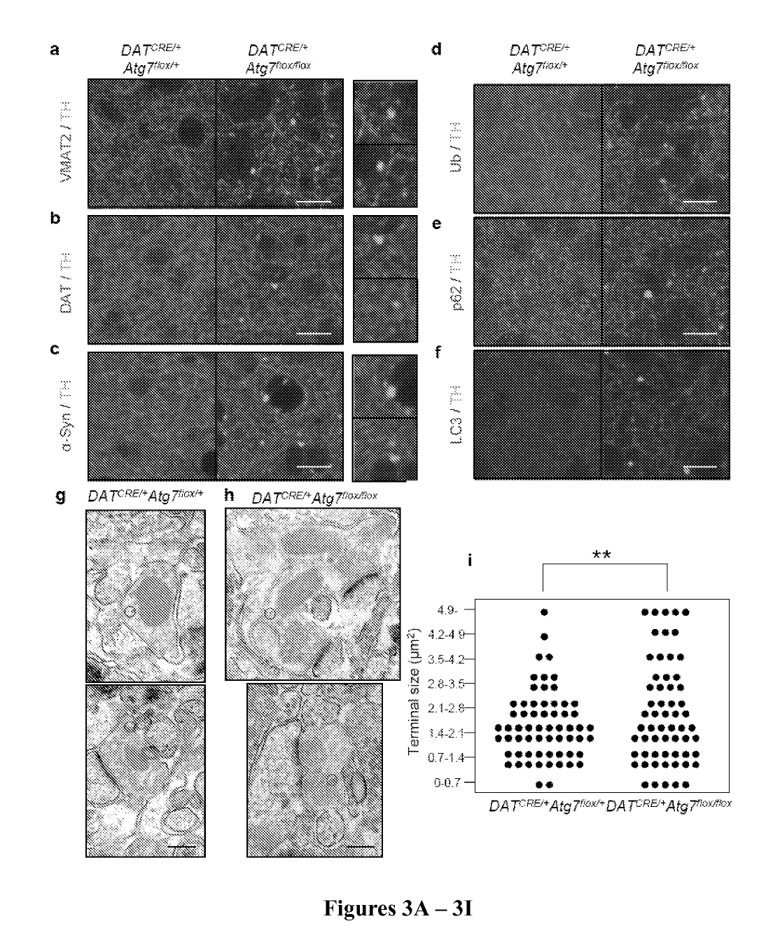

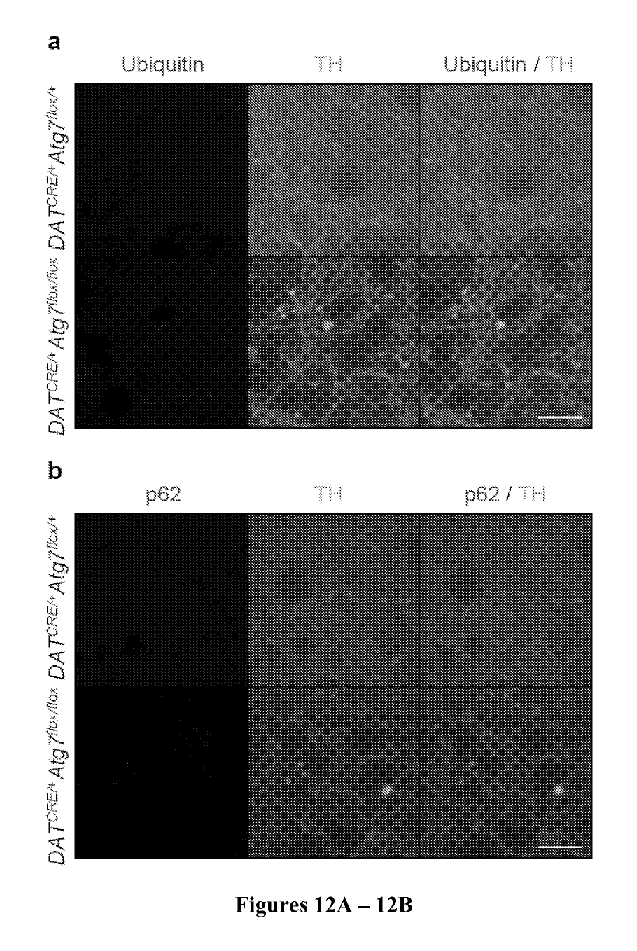

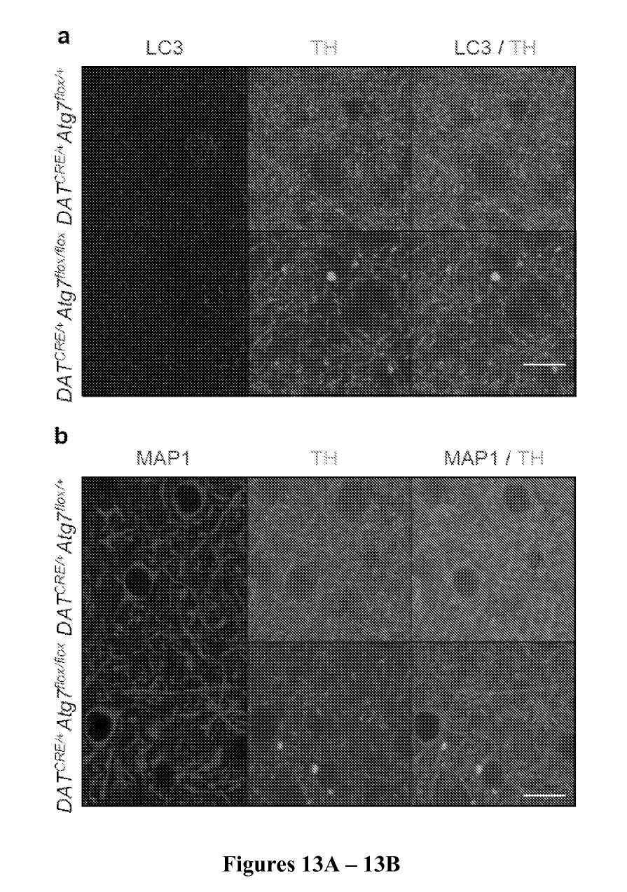

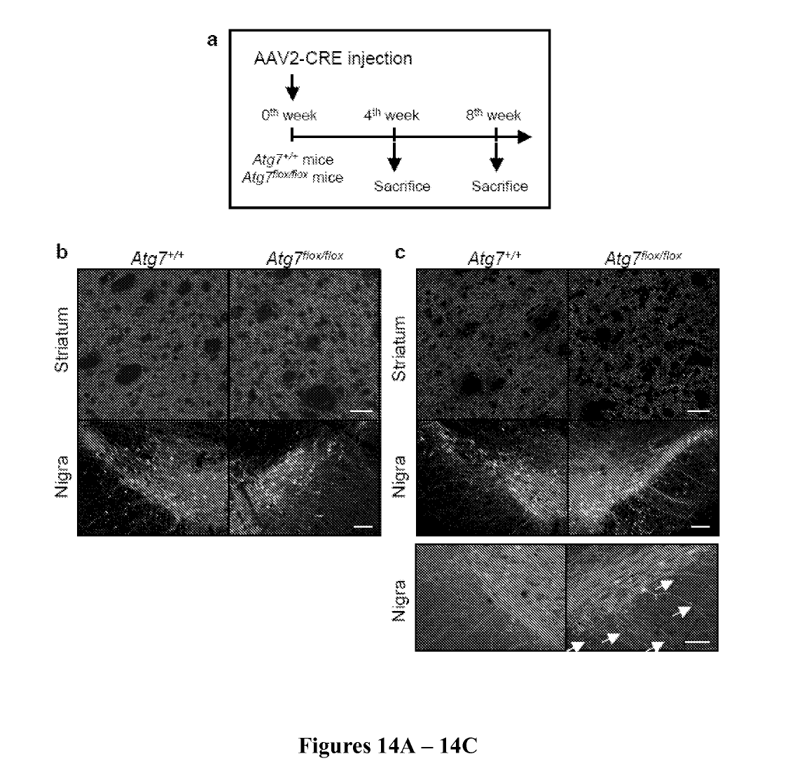

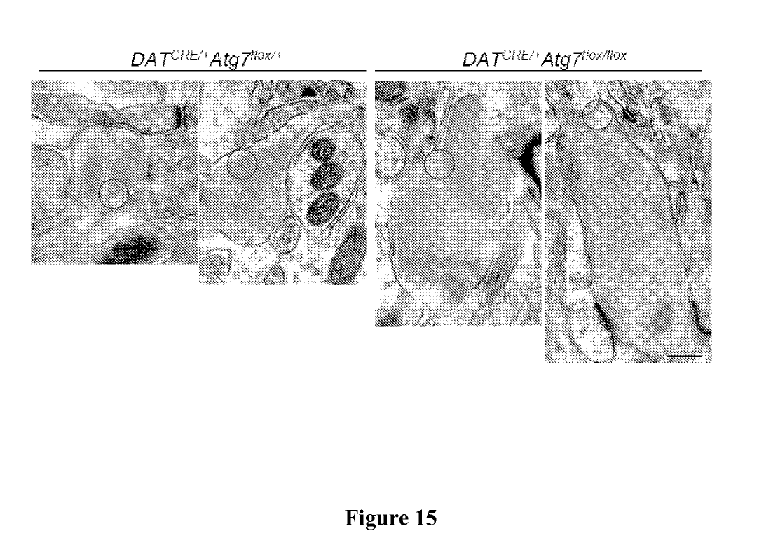

Grossly enlarged dopaminergic axonal structures were observed throughout the striatum of DAT^{CRE/+}Atg7^{flox/flox} mutant mice (FIGS. 3A-3F). These were apparent from 2 weeks of age (FIG. 11), and preceded other phenotypes observed in the mutant mice. The enlarged axonal structures stained positively with a panel of antibodies to dopaminergic presynaptic terminal components including the dopamine transporter (DAT), the vesicular monoamine transporter-2 (VMAT2), and α-Synuclein (FIGS. 3A-3C and 11). No inclusions were apparent within axonal terminals, and the enlarged dopaminergic terminals were not stained with antibodies to components of the protein degradation machinery such as ubiquitin, p62/SQSTM1 and LC3 (FIGS. 3D-3F, 12 and 13). Staining for a post-synaptic striatal dendritic marker, MAP1, appeared unaltered (FIG. 13). Consistent with these findings, immunoelectron microscopy for TH showed significant enlargement but otherwise normal morphology of autophagy-deficient striatal dopaminergic axonal terminals, including normal appearing presynaptic terminals, synaptic vesicles, and mitochondria (FIGS. 3G-3I and 15). This axonal change is not a consequence of developmental changes, as introduction of CRE recombinase by adeno-associate viral-mediated transduction in 2-month old Atg7^{flox/flox} mice recapitulates this phenotype (FIG. 14). Thus, the enlarged axonal terminals of Atg7-deficient mDNs that were observed appear qualitatively different from the dystrophic axonal swellings and proteinaceous and membranous inclusions reported in the context of autophagy loss in other CNS neuron populations ⁵⁻⁷. Axonal enlargement in Atg7-deficient mDNs may relate to the role of Atg7 in autophagy or to other unrelated functions.

### Atg7 Regulation of Axonal Terminal Size Through the PI3KI Pathway

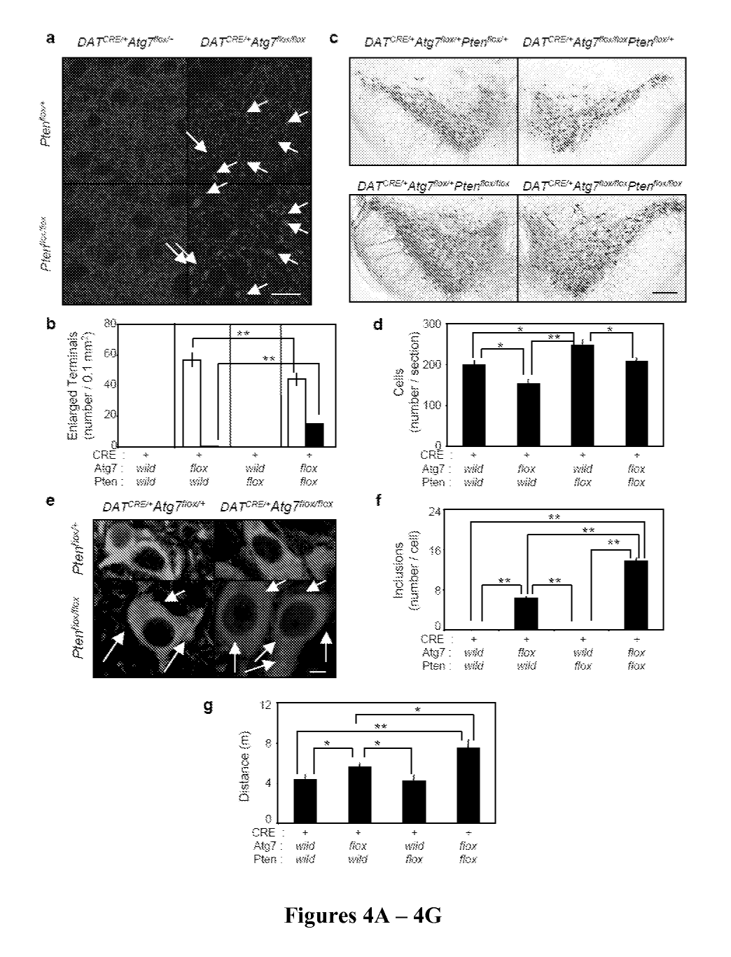

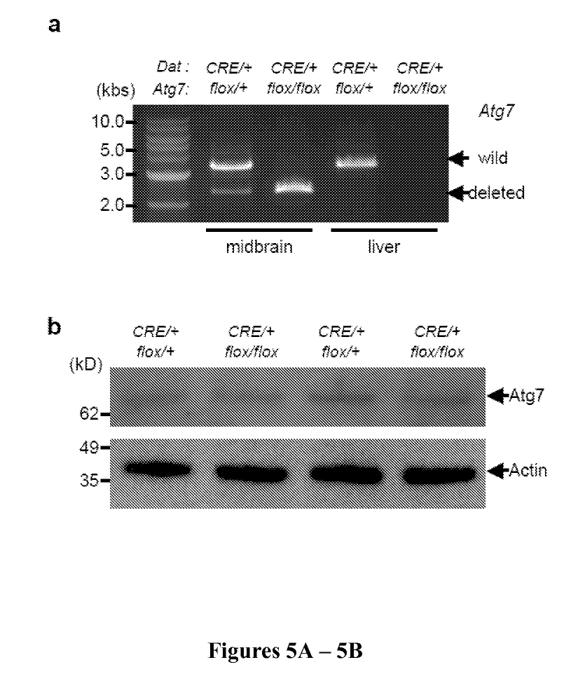

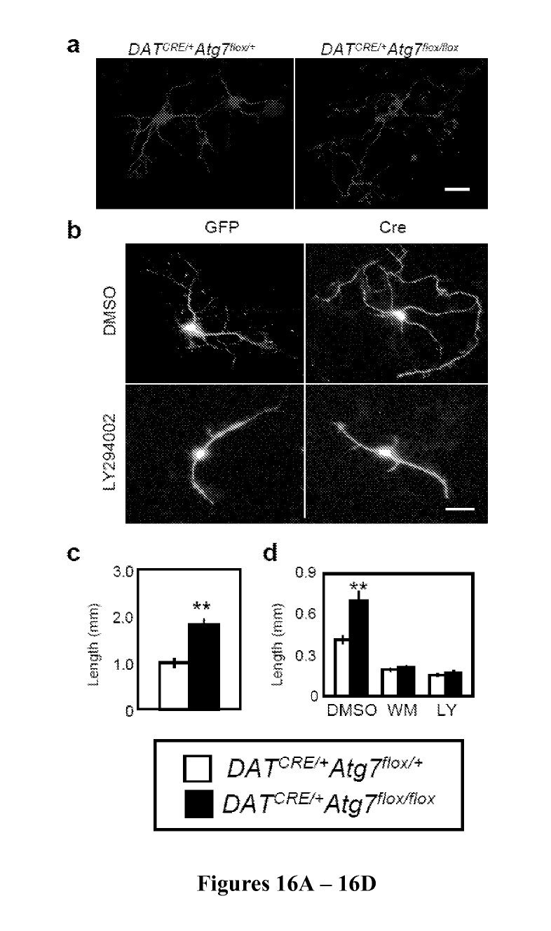

The early axonal enlargement of Atg7 deficient mDNs may be a consequence of altered axonal intracellular signaling. A key regulatory mechanism of axonal terminal size is the PI3KI pathway ^{14,15}, which is also an important regulatory pathway upstream of autophagy induction ^{2,16}. Experiments were designed to determine the role of the PI3KI signaling pathway in the context of mDN Atg7 deficiency. To this end, double mutant mice (DAT^{CRE/+}Atg7^{flox/flox}Pten^{flox/flox}) were generated with mDNs deficiency of both Atg7 and the Phosphatase and Tensin homologue (PTEN), a lipid phosphatase that dephosphorylates phosphatidylinositol (3,4,5)-trisphosphate (PI[3,4,5]P3) and thus antagonizes PI3K activity¹⁷. DAT^{CRE/+}Atg7^{flox/flox}Pten^{flox/flox} were compared to single mutants (DAT^{CRE/+}Atg7^{flox/flox} or DAT^{CRE/+}Pten^{flox/flox}) as well as control mice that harbor only the CRE transgene. In double mutant (DAT^{CRE/+}Atg7^{flox/flox}Pten^{flox/flox}) mice lacking both Atg7 and PTEN in mDNs, dopaminergic axonal terminals were much larger than those in single mutant DAT^{CRE/+}Atg7^{flox/flox} mice that lack only Atg7 (FIG. 4A), whereas dopaminergic axons deficient in PTEN alone (DAT^{CRE/+}Pten^{flox/flox}) appeared normal. These data suggest that Atg7 normally suppresses axonal terminal size, at least in part through inhibition of PI3K pathway regulation of terminal size. Additionally, PTEN pathway mediated-enlargement of terminals does not require the presence of Atg7 and therefore is not mediated strictly through the inhibition of autophagy. Consistent with this model, in vitro primary midbrain cultures from Atg7-deficient mice display increased neurite process length relative to controls (FIG. 16), and this phenotype is suppressed by the PI3KI inhibitors Wortmannin or LY294002. As PI3KI signaling is known to function upstream of and negatively regulate autophagy ²′¹⁶, these data show the presence of a role for PI3KI signaling downstream of Atg7 in the regulation of dopaminergic axonal terminal architecture.

PI3KI Pathway Activation Rescues the mDN Loss Associated with Atg7 Deficiency

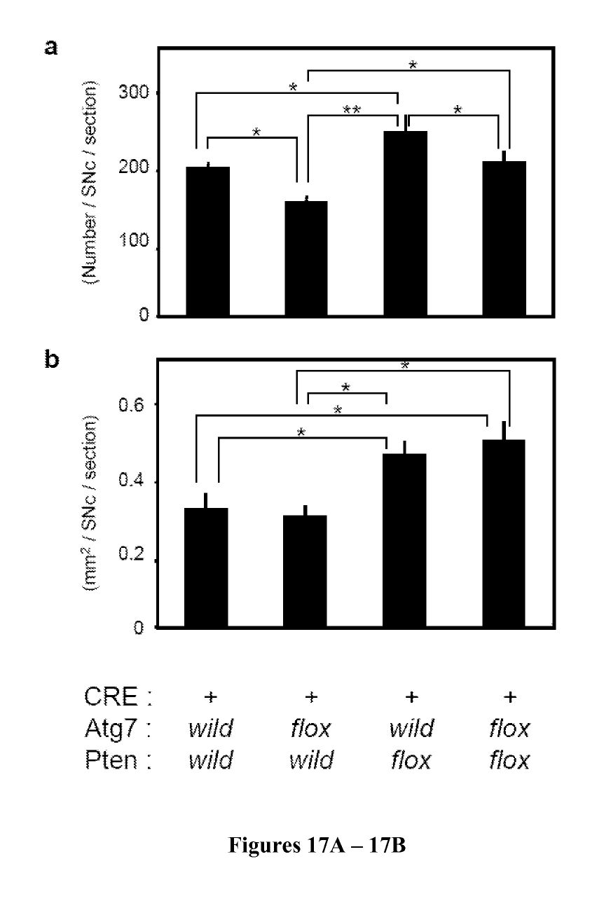

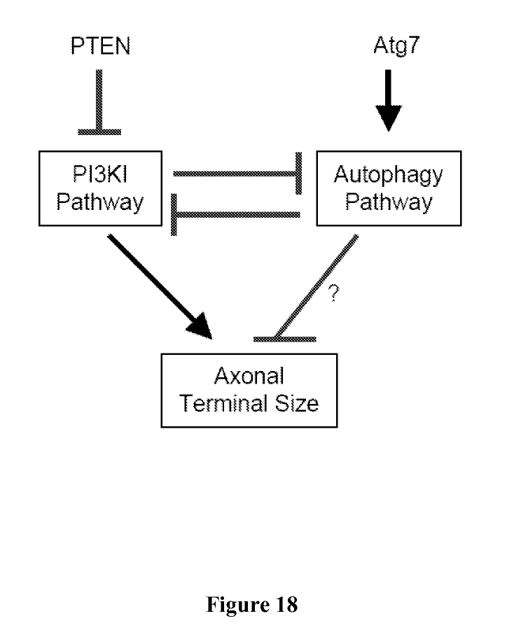

Experiments were designed to further probed the relationship of PI3KI signaling with autophagy in the context of Parkinson's disease-related phenotypes such as mDN loss and inclusion formation. Studies of PTEN-deficient (PI3KI pathway-activated) neurons elsewhere in the murine CNS have demonstrated that PI3KI induction leads to increased neuron size but does not appear to alter neuron number^{15,19}. In contrast, mice deficient in PTEN within midbrain dopamine neurons (DAT^{CRE/+}Pten^{flox/flox}) showed a significant increase in the number of midbrain dopamine neurons at 2 months of age in addition to increased size (FIGS. 4C-4D and 17). PTEN deficiency rescued loss of Atg7-deficient mDNs, as double mutants deficient in both Atg7 and PTEN within mDNs (DAT^{CRE/+}Atg7^{flox/flox}Pten^{flox/flox} displayed a normal number of mDN. Surprisingly, ubiquitin-positive aggregate formation in Atg7 deficient mDNs was significantly enhanced by the additional loss of PTEN (in DAT^{CRE/+}Atg7^{flox/flox} double mutants; FIGS. 4e and f). Thus, PI3KI pathway activation (in the context of PTEN deficiency) potentiates the formation of inclusions through a mechanism that is normally suppressed by autophagy. Furthermore, as activation of the PI3KI pathway leads to reduced cell loss but increased inclusion formation, these data suggest that such inclusions are not responsible for mDN loss associated with Atg7 deficiency, and may be protective.

Atg7 deficiency in mDNs modifies motor activity through PI3KI signaling mDNs regulate mammalian motor activity, and loss of mDN in PD is associated with motor dysfunction including both reduced activity (such as initiation of movements) and disinhibited movements (such as tremor at rest). Experiments were designed to determine the role of Atg7 and PTEN in dopaminergic neuron motor control. Mice deficient in Atg7 within mDNs (DAT^{CRE/+}Atg7^{floxflox}) display disinhibited activity in an open field (FIG. 4G), whereas mice deficient in PTEN within mDNs (DAT^{CRE/+}Pten^{flox/flox}) show normal activity. Double mutant mice (DAT^{CRE/+}Atg7^{flox/flox}) displayed a further increase in motor activity beyond the autophagy-deficient animals (DAT^{CRE/+}Atg7^{flox/flox}). These data correlate with the synergistic effect of PTEN deficiency and Atg7 loss in the regulation of axonal terminal size, but not with the antagonistic effect of PTEN and Atg7 deficiency in suppressing mDN loss. Thus, changes in motor behavior in Atg7 deficient mice are likely a consequence of altered dopaminergic axonal terminal morphology, rather than mDN cell loss.

In summary, these studies show that morphological changes at dopaminergic axonal terminals in the context of Atg7 deficiency are not simply a consequence of engorgement and dystrophic changes due to defective protein degradation, as has been described in other CNS neuron types ⁵⁻⁷. Rather, Atg7 regulates axonal terminal size within a complex feedback circuit with PI3KI signaling. Other signaling pathways downstream of Atg7 may play a role in the regulation of axonal size. Furthermore, whether Atg7 deficiency functions through autophagy changes to regulate axonal size, or whether this represents an unrelated function of Atg7 is unclear; it remains to be seen whether other essential autophagy components also function in the regulation of dopaminergic axonal size. The basis of the apparent cell type-specificity of Atg7 function at the axonal terminal is also of particular interest. The relationship of autophagy with the PI3KI pathway appears qualitatively different at the soma: activation of the PI3KI pathway suppresses Atg7 deficiency-associated mDN loss, but the two pathways function independently.

The pathological phenotype of aged Atg7-deficient mice is reminiscent of Parkinson's disease pathology, with large α-Synuclein positive, ubiquitin-positive inclusions. α-Synuclein does not appear to play a primary role in the nucleation of inclusions, as α-Synuclein immunostaining appears late. An alternative model is that α-Synuclein accumulation acts upstream of the PI3KI pathway to suppress autophagy in PD; consistent with this model, autophagic alterations have been described in patient pathology and in cell models^{2,3,19}, and α-Synuclein appears to inhibit a related degradation pathway termed chaperone-mediated autophagy²⁰.

This Example shows that selective deficiency of the essential autophagy component Atg7 in mDNs leads to the progressive loss of these cells and the presence of intracellular proteinaceous inclusions that mimic Parkinson's disease pathological changes. α-Synuclein is present but appears late in these inclusions. Disinhibition of the PI3KI pathway due to PTEN deletion protects mDNs from death in the context of autophagy deficiency. PTEN deletion in autophagy-deficient mDNs increases inclusion formation, arguing against a toxic role for the inclusions. Axonal terminals of ATG7-deficient mDNs do not appear dystrophic but are dramatically enlarged due to disinhibition of the PTEN pathway. Altered motor activity in ATG7-mutant mice correlates with axonal morphology rather than mDN loss. These data reveal a complex feedback circuitry between autophagy and the PI3KI pathway in mDN inclusions, axonal architecture, function, and survival.

### Exemplary Methods

The following are non-limiting examples of methods that may be used in connection with the disclosed invention.

Animal. DAT^{CRE/+} mice, Atg7^{flox/flox} mice, and Pten^{flox/flox} mice used in this study were generated previously. The DAT^{CRE/+} mice were obtained from Dr. Hen (Columbia University). The Atg7^{flox/flox} mice were obtained from Dr. Tanaka (Tokyo Metropolitan Institute of Medical Science). The Pten^{flox/flox} mice were purchased from Jackson Laboratories. DAT^{CRE/+}Atg7^{flox/flox} mice were obtained from the breeding of DAT^{CRE/+}Atg7^{flox/+}×Atg7^{flox/flox} or DAT^{CRE/+}Atg7^{flox/+}×Atg7^{flox/wild}. DAT^{CRE/+}Atg7^{wild/+} or DAT^{CRE/+}Atg7^{flox/+} mice from the same litter were used as the controls. All animals were maintained in the animal facility of the Columbia University Medical Center, with dry food pellets and water available ad libitum. All of the experimental protocols were approved by the Institutional Animal Care and Use Committees.

Genomic PCR. Genomic PCR was done to determine mouse genotypes and to detect Cre-mediated DNA recombination. Genomic DNA extracted from mouse tails were amplified by PCR for genotyping. For PCR genotyping, the following primers were used: Cre sense primer, 5′-TGTCCAATTTACTGACCGTACACCA-3′ (SEQ ID NO:1), Cre antisense primer, 5′-CAGTACGTGAGATATCTTTAACCCT-3′ (SEQ ID NO:2); Atg7 (wild) sense primer, 5′-TGCTCTGTGAACTGCCCTGTTT-3′ (SEQ ID NO:3); Atg7 (wild) antisense primer, 5′-TGTTCCTGTGCACTGCCTCATT-3′ (SEQ ID NO:4); Neo sense primer, 5′-CTTGGGTGGAGAGGCTATTC-3′ (SEQ ID NO:5); Neo antisense primer, 5′-AGGTGAGATGACAGGAGATC-3′ (SEQ ID NO:6); Pten sense primer, 5′-ACTCAAGGCAGGGATGAGC-3′ (SEQ ID NO:7); Pten antisense primer, 5′-AGGGAGGATGAATCTGTGCA-3′ (SEQ ID NO:8). Genomic DNA extracted from midbrains or livers were amplified by PCR for Cre-mediated DNA recombination. For DNA recombination, the following primers were used: Primer (Hind-Fw), 5′-TGGCTGCTACTTCTGCAATGATGT-3′ (SEQ ID NO:9); Primer (96-121c), 5′-TTAGCACAGGGAACAGCGCTCATGG-3′ (SEQ ID NO:10).

Western Blotting. Midbrain tissues (4-7 mg) were dissected from mouse brains and homogenized in 10 times volume of ice-cold RIPA buffer (50 mM Tris-HCl [pH8.0], 150 mM NaCl, 1% NP-40, 0.5% deoxycholate, 0.1% SDS, and proteinase inhibitor cocktail [P8340, Sigma-Aldrich, St. Louis, Mo.]). The homogenized tissues were kept on ice for 30 min and centrifuged for 10 min at 4° C. The supernatants (5 μg per lane) were used for SDS-PAGE and western blotting. The membranes were incubated in the buffer (Tris-buffered saline containing 5% non-fat skim milk and 0.1% Tween20) containing primary antibody at 4° C. overnight and in the buffer containing secondary antibody at room temperature for 1 hour. Primary antibodies to detect Atg7, and beta-actin were rabbit anti-APG7L (1:100, ABGENT, San Diego, Calif.), and mouse anti-actin (1:300, A3853, Sigma-Aldrich, St. Louis, Mo.). Secondary antibodies were horseradish peroxidase-conjugated anti-rabbit-IgG (1:1000, 111-036-045, Jackson Immunoresearch, West Grove, Pa.) and anti-mouse IgG (1:1000, 115-036-062, Jackson Immunoresearch, West Grove, Pa.). The membranes were soaked for 5 min in SuperSignal West Dura (34075, PIERCE, Rockford, Ill.) for Atg7 and ECL Western blotting Detection Reagents (RPN2106, GE Healthcare) for actin, and exposed to X-ray film for a few minutes.

Histology. Mice were perfused and fixed in 50 ml of ice-cold 4% paraformaldehyde and post-fixed in the same solution at 4° C. for overnight. Fifty-μm coronal brain sections were cut by vibratome (VT1000S, Leica, Bannockburn, Ill.). To visualize TH-positive neurons, polyclonal anti-TH antibody made in sheep was used (P60101, Pel-Freez, Rogers, Ak.) at the dilution of 1:250. For chromogenic staining, sections were treated in 3% H₂O₂ for 10 min and blocked in PBS solution containing 5% normal donkey serum and 0.05% TritonX-100 (blocking solution) for 30 min. After blocking, sections were incubated in antibody solution containing 1% normal donkey serum and 0.05% TritonX-100 at 4° C. for three overnight. The following steps were according to Vectastain Elite ABC kit (Sheep IgG, PK-6106, Vector Laboratories, Burliname, Calif.) and DAB substrate kit (SK-4100, Vector Laboratories, Burliname, Calif.). For fluorescent staining, sections were blocked in blocking solution for 30 min and incubated in antibody solution at 4° C. for four overnight. After washing, the sections were incubated with secondary antibody solution (1:1000, Cy3-conjugated anti-sheep IgG, 713-166-147, Jackson Immunoresearch, West Grove, Pa.). For multiple stainings with TH-antibody, the sections were first stained with other primary antibodies (4° C., overnight) and corresponding secondary antibodies conjugated with Cy3 (1:1000, 711-166-152, -150, -148, Jackson Immunoresearch, West Grove, Pa.). Then, the stained sections were incubated in sheep anti-TH antibody and FITC-conjugated anti-sheep IgG antibody to prevent the cross-reaction to sheep anti-TH antibody. The antibodies used here were rabbit anti-Cre (1:3000, PRB-106C, Covance, Emeryville, Calif.), rabbit anti-ubiquitin (1:100, Sigma-Aldrich, St. Louis, Mo.), guinea pig anti-p62 (1:100, 03-GP62-C, American Research Products, Belmont, Mass.), rabbit anti-LC3B (1:2000, NB600-1384, Novus Bio, Littleton, Colo.), rabbit anti-VMAT2 (1:200, AB1767, Chemicon, Temecula, Calif.), rat anti-DAT (1:1000, MAB369, Chemicon, Temecula, Calif.) and rabbit anti-alpha-synuclein C-20 (1:100, sc-7011-R, Santa Cruz, Santa Cruz, Calif.). Single staining was performed without anti-TH staining and double staining without primary antibodies as the negative controls.

Morphometry. For morphometric analyses of TH-positive neurons, every third 50-μm vibratome section from frontal to dorsal midbrain was immunostained with sheep anti-TH antibody. Pictures were taken at 40× magnification. The number of TH-positive neurons was counted and their area was measured by an observer blind to genotype, using Image-J software (NIH). The cell density was calculated as cell number/area.

HPLC. Mice were put to sleep in CO₂, and the nigral and striatal tissues were carefully dissected (nigra, 4-6 mg; striatum, 9-12 mg). The tissues were put into 500 μl of ice-cold 0.1M percholoric acid solution containing 50 ng/ml DHBA, homogenized and sonicated 2 times for 10 sec on ice. 50-μl of homogenates was saved, and the protein concentration was measured by Bradford assay. Residual homogenates were centrifuged at 14,000 rpm at 4° C. for 20 min, and the supernatants were collected. Ortho-phosphoric acid and metabisulfate were added to the supernatants into the final concentration of 8.8% and 0.22 mg/ml, respectively. Concentration of dopamine, DOPAC, HVA, 5HT, and 5HITT in the supernatants was measured by HPLC. The concentrations were standardized by protein concentration.

Electron Microscopy. Anesthetized mice were perfused and fixed in 35 ml of ice-cold phosphate buffer saline (PBS) containing 4% paraformaldehyde and 0.5% glutaralaldehyde. The brains were post-fixed at 4° C. for 2 hours, and sectioned at 80 μm on a vibratome. The sections were treated in 50 mM glycine/PBS at room temperature to inactivate aldehyde and washed in PBS. To enhance penetration, the sections were incubated in 2.5% glycerol and 25% sucrose in PBS for 15 min, mounted in OCT compound, dipped in liquid nitrogen, and immersed in PBS at room temperature. They were blocked in PBS containing 5% BSA and 5% normal rabbit serum at room temperature for 30 min and washed in incubation buffer (0.1% Aurion BSA-c™/PBS, #25557, Electron Microscopy Sciences, Hatfield, Pa.) for 5 min. They were incubated at 4° C. for 4 overnight in incubation buffer containing primary antibody. The dilution of rabbit anti-tyrosine hydroxylase antibody is 1:250 (P40101, Pel-Freez, Rogers, Ak.). After the incubation, the sections were washed in incubation buffer 3 times for 5 min. The sections were incubated in the ultra-small gold-conjugated goat anti-rabbit IgG antibody (#25100, Electron Microscopy Sciences, Hatfield, Pa.) at room temperature for 2 hours. The dilution of the secondary antibody is 1:50. The sections were washed in incubation buffer for 5 min, 6 times and in PBS for 5 min, 3 times. The sections were post-fixed in 2% glutaralaldehyde/PBS. After 5 times washes in distilled water, the silver enhancement was done by using Aurion R-Gent SE-EM (#25521, Electron Microscopy Sciences, Hatfield, Pa.) for electron microscopy and Aurion R-Gent SE-LM (#25520, Electron Microscopy Sciences, Hatfield, Pa.) for light microscopy.

AAV2-CRE/GFP infection. Two μl of 10× diluted AAV2 was injected into the right midbrains of anesthetized 2-month-old Atg7^{+/+} or Atg7^{flox/flox} mouse on the stereotaxic flame apparatus. The injection site is 3.1 mm (X-axis), 1.1 mm (Y-axis) and 4.4 mm (Z-axis) from the bregma. For control experiment, AAV2-GFP was injected into the contralateral site of the midbrain. The mice were perfused and fixed 4- or 8-weeks after the injection.

Cell Culture. For midbrain culture, midbrain tissues were dissected from newborn mice (postnatal 0 day). The tissues were minced and digested in isolation medium containing 0.25% trypsin and ascorbic acid at 37° C. for 30 min. After the incubation, the digestion was stopped by 3% fetal bovine serum and 0.25% Dnase I. The tissues were triturated 20 times by glass-pipette and filtrated by cell strainer. They were centrifuged at 1,200 rpm for 5 min, and the supernatants were removed. The cells were suspended in plating medium containing 10% fetal bovine serum and plated on poly-D-lysine and laminin-coated 24-well plates. On the next day, the plating medium was replaced by culture medium containing 1% fetal bovine serum and antimitotic agent. The cells were kept on 37° C. for three more days. For cortical culture, cortical tissues were dissected from newborn mice (postnatal 0 day). The tissues were minced and digested in isolation medium containing 0.25% trypsin at 37° C. for 30 min. After the incubation, the digestion was stopped by 3% fetal bovine serum and 0.25% Dnase I. The tissues were triturated 20 times by glass-pipet and filtrated by cell strainer. They were centrifuged at 1,200 rpm for 5 min, and the supernatants were removed. The cells were suspended in plating medium containing 10% fetal bovine serum and plated on poly-D-lysine and laminin-coated 24-well plates. On second day, the cells were transfected with GFP or Cre expression plasmids. To transfect the plasmids, the cells were washed in 500 μl DMEM and added 30 μl of DNA/Ca/Phosphate complex (2 μg DNA/3.75 μl 1M CaCl₂/15 μl 2× HBSS). The cells were incubated at 37° C. for 20 min, and the medium was replaced by plating medium the plating medium containing 10% fetal bovine serum. On third day, the medium was replaced by culture medium containing 1% fetal bovine serum, antimitotic agent and PI3K inhibitor. The cells were kept on 37° C. for two more days.

Mouse Behavior. 4- to 6-month-old male mice were used for open field test and the tests were carried out during the dark cycle of their circadian rhythm. The mice were placed on an experimental room one hour prior to the test. The activities of the animal were monitored automatically by beam interruption for 30 min in the novel open field box of 17.0″×17.0″ (MED-OFA-RS, Med Associates Inc., St Albans, Vt.) or 11.0″×11.0″ (MED-OFA-MS, Med Associates Inc., St Albans, Vt.). As the first trial, DAT^{CRE/+}Atg7^{flox/+} mice and DAT^{CRE/+}Atg7^{flox/flox} mice (n=19 and 17, respectively) were analyzed in the box of 17.0″×17.0″. As the second trial, DAT^{CRE/+}Atg7^{flox/+}Pten^{flox/+} mice, DAT^{CRE/+}Atg7^{flox/flox}Pten^{flox/+} mice, DAT^{CRE/+}Atg7^{flox/flox}Pten^{flox/+} mice, and DAT^{CRE/+}Atg7^{flox/flox}Pten^{flox/flox} mice (n=12, 10, 9, and 11, respectively) were analyzed in the box of 11.0″×11.0″ (FIG. 4G). Data was analyzed by using Activity Monitor (Med Associates Inc., St Albans, Vt.). Same results were obtained from two different trials on the locomotor activity of DAT^{CRE/+}Atg7^{flox/+} mice and DAT^{CRE/+}Atg7^{flox/flox} mice.

Statistical Analysis. All of the comparisons were made with the Mann-Whitney U-test (for two samples) or non-repeated measures ANOVA (for multiple samples). The values are expressed as the means±S.E. A p value less than 0.05 is considered significant.

### Example 2

### Protection of Neurons by Suppression of PTEN or Gsk3β, or Activation of AKT in a Parkinson's Disease Cell Model

Mutations in leucine-rich repeat kinase-2 (LRRK2 (herein), PARK8, dardarin) (GenBank Accession No: NM_{—}025730) underlie an autosomal dominant, inherited form of P) that mimics all of the clinical features of the common sporadic form of PD. Mammalian LRRK2 regulates neurite maintenance and neuronal survival. Neurons that express PD-associated mutant forms of LRRK2 display reduced process length and complexity, Tau-positive protein aggregates, and, ultimately, apoptotic cell death. NMDA receptor antagonists and antioxidants inhibit LRRK2 mutant-associated phenotype, consistent with a role for glutamate excitotoxicity in PD. LRRK2 clinical mutations, including G2019S, lead to a reduction in the length and complexity of cortical neuron processes. In contrast, suppressing LRRK2 activity, for example by using a dominant negative allele or RNA interference (RNAi), leads to an increase in neuron process length and complexity. International Patent Application Publication No. WO 2007/124096, which is incorporated by reference herein, describes PD-associated LRRK2 mutations and cell-based assays used in this Example.

The PTEN/PI3K/AKT/Gsk3β signal cascade plays a central role in the regulation of neuronal length and complexity, and also functions downstream of glutamate excitotoxicity. The interaction between LRRK2 and PTEN/AKT/GSK3β was investigated using the methods described in International Patent Application Publication No. WO 2007/124096.

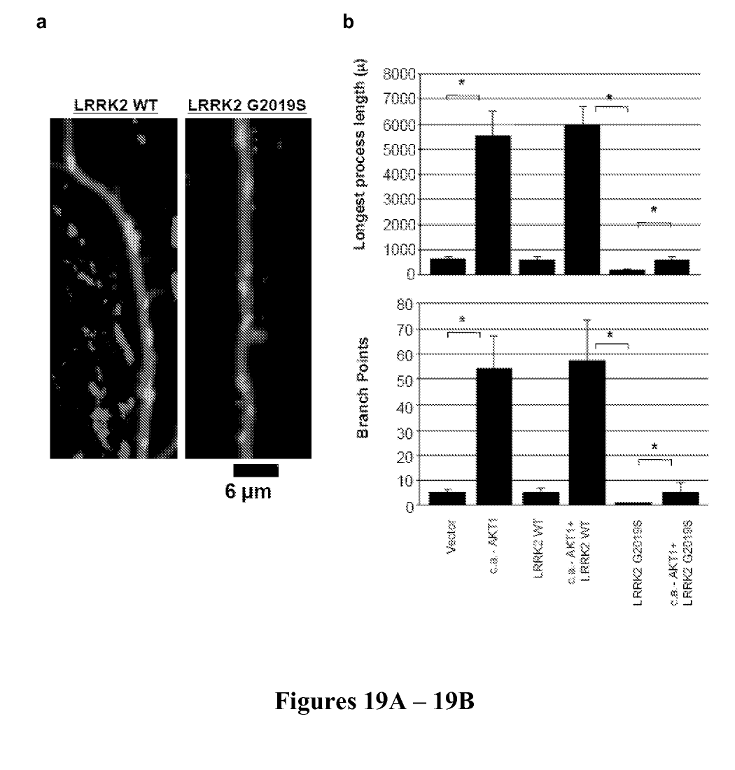

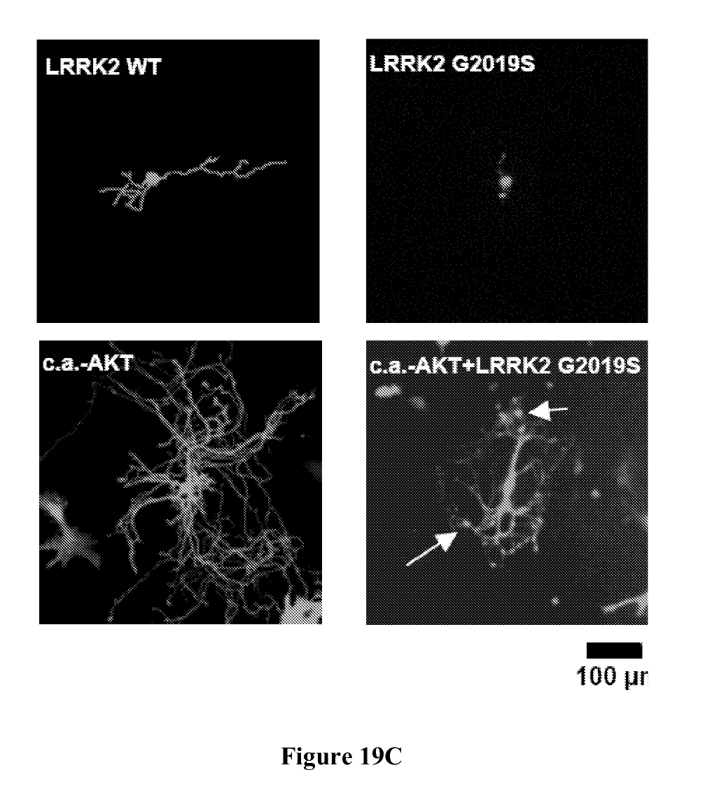

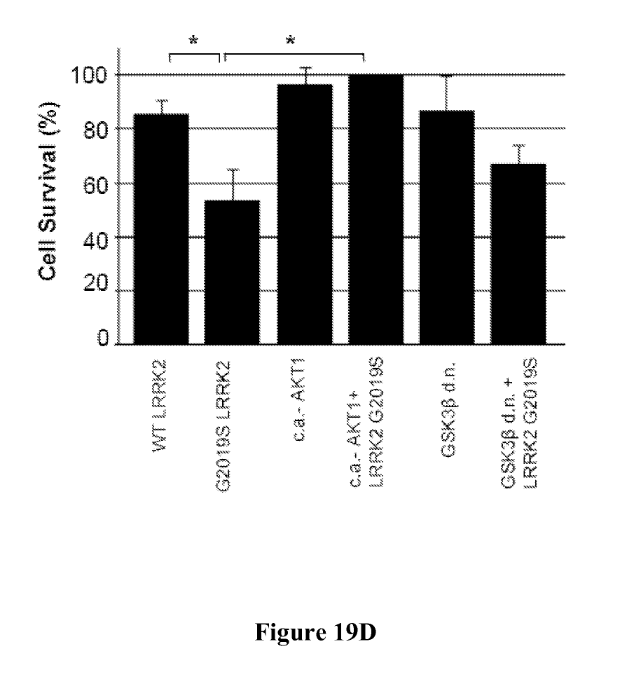

Given the known role of the AKT kinase signaling pathway in neuron survival in the context of glutamate excitotoxicity and oxidative stress (Datta et al., 1999), as well as the central role of this pathway in the regulation of neurite length and complexity (Shi et al., 2003), experiments were designed to test whether the AKT signaling pathway interacts with LRRK2 induction. Consistent with this model, overexpression of a constitutively active form of AKT1 (Datta et al., 1997) antagonizes the toxicity of G2019S LRRK2 in co-transfection assays in terms of both neuronal morphology and survival (FIGS. 19A-19D). In contrast, the spheroid aggregate phenotype does not appear to be rescued by AKT1 activity (FIG. 19C). Similar results were observed by co-transfection of a dominant negative form of GSK-3β, a downstream target of AKT that is inhibited by AKT activation (FIG. 19D).

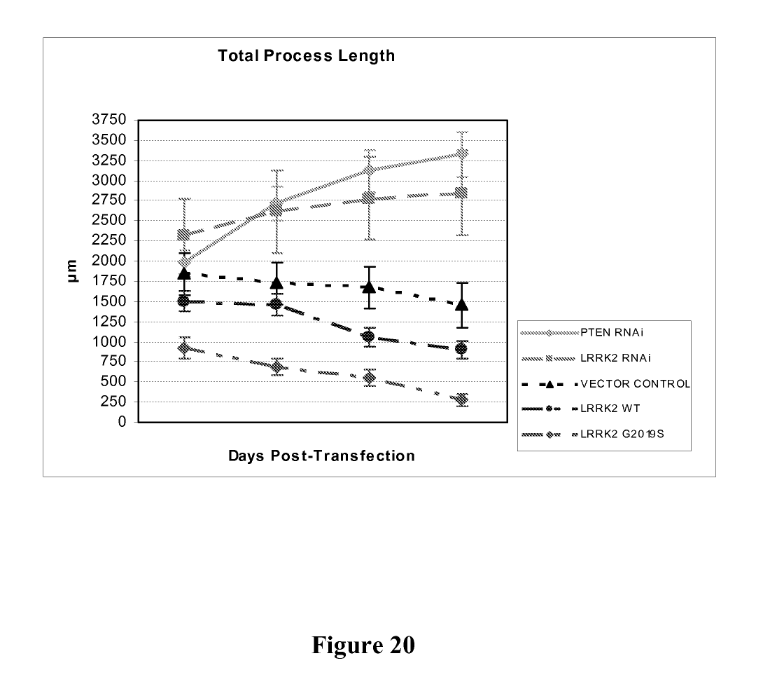

The Phosphatase and Tensin homologue (PTEN), is a lipid phosphatase that dephosphorylates phosphatidylinositol (3,4,5)-trisphosphate (PI[3,4,5]P3) and thus antagonizes PI3K activity. Data presented in Example 1 show the presence of a role for PI3KI signaling downstream of Atg7 in the regulation of dopaminergic axonal terminal architecture. As shown in FIG. 20, in a cell model of PD, genetic suppression of PTEN by RNAi prevents neurite loss.

### Example 3

### Additional Studies Using Autophagy-Deficient Mouse Model

This Example provides additional data from experiments conducted using the autophagy-deficient mouse model described in Example 1.

Postnatal mDN specific deletion of ATG7 in mice. B6×CBA mice with homozygous floxed alleles of ATG7 have been described. Briefly, these mice have LoxP sites flanking exon 14 of the ATG7 gene (herein the ATG7^{flox} allele), encoding the critical active site of the enzyme. These mice were crossed with dopamine transporter (DAT)-CRE mice that express the Cre recombinase specifically within midbrain dopamine neurons under the regulation of the dopamine transporter gene regulatory elements. (The DAT transporter is also expressed in other CNS dopaminergic cells, but at much lower levels, and thus the specificity of expression is not absolute. However, it does closely mimic the pattern of CNS cell loss seen in PD.) F1 animals were genotyped using standard techniques, and the double heterozygous animals were backcrossed to the ATG7^{flox/flox} parents to generate DAT:CRE^{+/-}; ATG7^{flox/flox} animals and littermates controls. These mice harbor a deletion in ATG7 specifically in midbrain dopamine neurons. Excision of loxP-sequence flanked ATG7 by Cre recombinase was detected in genomic midbrain DNA of mutant mice and the expression of Cre recombinase was detected only in TH-positive midbrain neurons (FIG. 5A). Expression of the Cre transgene was detected throughout all mDNs but not in other cell types (FIG. 1A). Cathepsin-B, a marker for lysosomes, was prominent in TH-positive cells of control mice, but absent in the mutant mice, consistent with a defect in the autophagy/lysosomal pathway.

Preliminary analysis of DAT:CRE^{+/-}; ATG7^{flox/flox} mice. DAT:CRE^{+/-}; ATG7^{flox/flox} mice were analyzed in comparison to DAT:CRE^{+/-}; ATG7^{flox/+} littermates by several measures. First, immunohistochemical analysis of the mice was performed in 2 week-old, 1 month-old, 3 month-old and 6 month-old animals, with an antibody for tyrosine hydroxylase (TH), the rate-limiting enzyme for dopamine synthesis. These studies revealed a progressive loss of TH-positive axonal processes in the striatum and of mDN cell bodies in the SN beginning at 1 month of age, as quantified by TH immunohistochemistry (FIG. 1B; a 60% loss +/−6% SEM).

Numerous TH-positive aggregates were apparent in ATG7 mutant substantia nigra dopaminergic cell bodies and dendrites at the 3-month and 6-month time points, whereas these were never seen in controls. These aggregates stained with antibodies to components of protein degradation machinery pathways—ubiquitin, p62/SQSTM1-consistent with defective protein degradation (FIGS. 2A-2B). The morphology and localization of these aggregates progressed over time. In 1-month old mutants, aggregates were relatively small and localized to the cell body of TH-positive neurons, whereas at later time points—up to 1-year old mutants—aggregates were greatly enlarged and extended into dendrites. Furthermore, α-synuclein-positive, ubiquitin-positive aggregates were apparent in 6-month old mutants (FIGS. 2D-2E), whereas α-synuclein-positive aggregates were not observed at earlier time points. These inclusions are reminiscent of the Lewy body inclusions that typify Parkinson's disease pathology. Consistent with this, electron microscopic examination revealed that aggregates harbored both filamentous and vesicular elements akin to Lewy body inclusions (FIG. 2F).

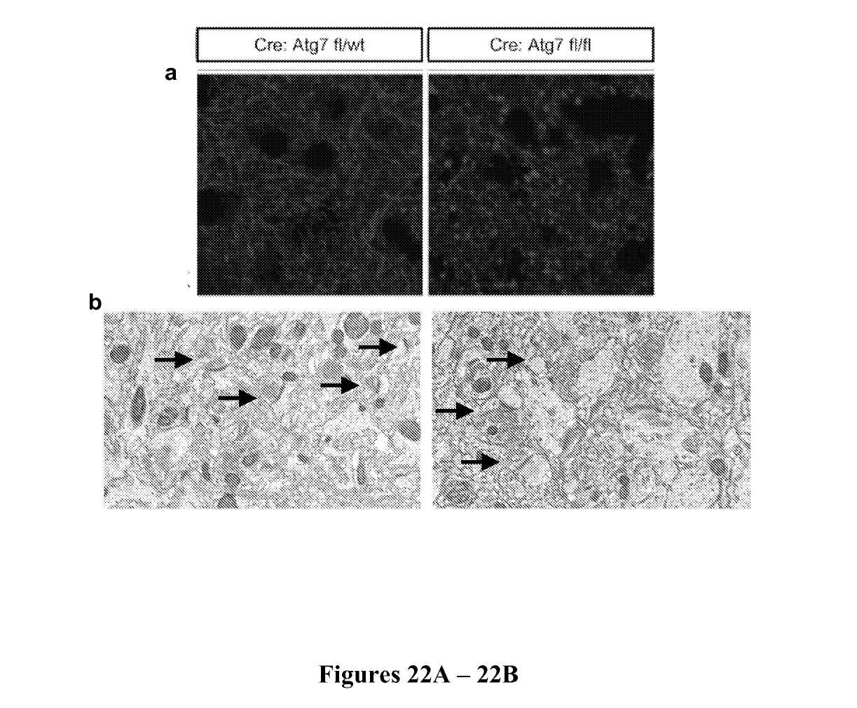

Experiments were designed to further define the enlarged dopaminergic axonal structures observed in the ATG7 mutant striatum (FIG. 22A). These structures were stained positively with a panel of antibodies to dopaminergic axonal presynaptic terminal components including the dopamine transporter (DAT), the vesicular monoamine transporter-2 (VMAT2), and alpha-synuclein. The enlarged dopaminergic terminals were not stained with antibodies to components of the protein degradation machinery such as ubiquitin and p62/SQSTM1, and no aggregates were apparent within axonal terminals. These findings argue against a model whereby the enlarged dopaminergic structures result from defective protein degradation leading to engorged terminals. Consistent with this, electron microscopic examination of axonal terminals in ATG7 mutant striatum did not reveal inclusions or multivesicular bodies. Rather, axon terminals were significantly increased in size but otherwise morphologically normal (FIG. 22B).

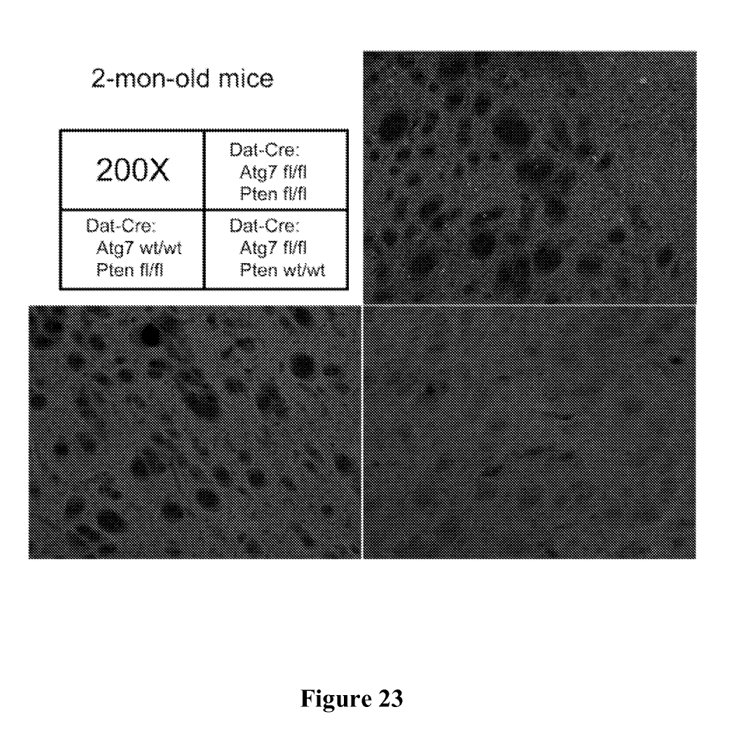

Analysis of DAT:CRE^{+/-}; PTEN^{flox/flox} mice. A key upstream regulatory pathway for autophagy, which is also implicated in Parkinsonism, is the PI3K/PTEN/AKT pathway. Analyses of mice with mutations in this pathway were conducted. PTEN −/− knockout mice display early embryonic lethality, and therefore conditional knockout mice were generated using the CRE:LoxP system to obtain animals with a specific deletion of PTEN within dopamine neurons in the midbrain. The PTEN^{flox/flox} mice were obtained from Jackson laboratories and crossed to DAT:CRE mice to generate DAT:CRE^{+/-}; PTEN^{flox/flox}. The mating scheme is essentially as described above for the ATG7 allele. These mice were then analyzed by IHC for TH at time points of 2 weeks, 1 month, and 6 months. PTEN knockout mice show a significantly increased number of mDNs (approximately 30%+/−4% SEM) and significantly increased neuron size and neurite process arbor complexity at 1 month of age and later. Prior studies have shown a role for PTEN in the regulation of neuronal size, but not cell number. Mice with deletion of both PTEN and ATG7 in mDNs were generated by standard crossing. These mice display rescue of the ATG7 mDN loss phenotype at 6 months, indicating that PTEN can function downstream of autophagy deficiency (FIG. 4C). In the striatal axonal terminals, PTEN loss alone led to no alteration but PTEN loss in the context of ATG7 loss markedly enhanced the enlargement of axonal terminals (FIG. 23). This indicates that PTEN can modify dopaminergic axonal terminal morphology downstream of ATG7 action.

αSyn transgenic mice. A hallmark of PD pathology is the presence of LB aggregates, which are composed largely of αSyn protein and ubiquitin. As noted, a prominent feature of the DAT:CRE^{+/-}; ATG7^{flox/flox} mice is a similar accumulation of αSyn positive inclusions. Interestingly, αSyn is believed to be degraded in part through the autophagy pathway, consistent with the observed accumulation of αSyn in the absence of autophagy. Additionally, there is a report of αSyn degradation through a related pathway, termed chaperone-mediated autophagy. To more specifically address the role of autophagy in αSyn degradation and toxicity in vivo, animals have been generated that overexpress Parkinsonism-associated mutant form of αSyn in the context of autophagy deficient dopamine neurons. Briefly, αSyn transgenic animals were obtained that harbor a human A53T mutant form of αSyn under the control of the murine prion promoter. These animals were crossed into the DAT:CRE^{+/-}; ATG7^{flox/flox} genetic background using standard mating approaches to obtain αSyn(A53T)+/−; DAT:CRE^{+/-};) ATG7^{flox/flox} animals as well as control littermates. Genotyping was performed by tail biopsy and PCR analysis on genomic DNA using standard techniques. These animals survive to at least 6 months of age.

Although the invention has been described and illustrated in the foregoing illustrative embodiments and examples, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

### REFERENCES

¹ A. E. Lang and A. M. Lozano, N Engl J Med 339 (15), 1044 (1998).
² R. A. Nixon, Trends in neurosciences 29 (9), 528 (2006).
³ P. Anglade, S. Vyas, F. Javoy-Agid et al., Histol Histopathol 12 (1), 25 (1997).
⁴ B. Levine and G. Kroemer, Cell 132 (1), 27 (2008).
⁵ M. Komatsu, S. Waguri, T. Chiba et al., Nature 441 (7095), 880 (2006).
⁶ M. Komatsu, Q. J. Wang, G. R. Holstein et al., Proceedings of the National Academy of Sciences of the United States of America 104 (36), 14489 (2007).
⁷ J. Nishiyama, E. Miura, N. Mizushima et al., Autophagy 3 (6), 591 (2007); T. Hara, K. Nakamura, M. Matsui et al., Nature 441 (7095), 885 (2006).
⁸ D. J. Klionsky, Nature 441 (7095), 819 (2006).
⁹ N. Chuhma, H. Zhang, J. Masson et al., J Neurosci 24 (4), 972 (2004); J. Kim, K. Inoue, J. Ishii et al., Science (New York, N.Y. 317 (5842), 1220 (2007).
¹⁰ M. Komatsu, S. Waguri, T. Ueno et al., J Cell Biol 169 (3), 425 (2005).
¹¹ S. Pankiv, T. H. Clausen, T. Lamark et al., The Journal of biological chemistry 282 (33), 24131 (2007); M. Komatsu, S. Waguri, M. Koike et al., Cell 131 (6), 1149 (2007).
¹² L. S. Formo, Advances in neurology 45, 35 (1987).
¹³ B. I. Giasson, J. E. Duda, S. M. Quinn et al., Neuron 34 (4), 521 (2002).
¹⁴ H. Jiang, W. Guo, X. Liang et al., Cell 120 (1), 123 (2005).
¹⁵ C. H. Kwon, B. W. Luikart, C. M. Powell et al., Neuron 50 (3), 377 (2006).
¹⁶ S. Arico, A. Petiot, C. Bauvy et al., The Journal of biological chemistry 276 (38), 35243 (2001); A. Petiot, S. Pattingre, S. Arico et al., Cell structure and function 27 (6), 431 (2002); T. Noda and Y. Ohsumi, The Journal of biological chemistry 273 (7), 3963 (1998); D. D. Sarbassov, S. M. Ali, and D. M. Sabatini, Current opinion in cell biology 17 (6), 596 (2005).
¹⁷ M. L. Sulis and R. Parsons, Trends in cell biology 13 (9), 478 (2003).
¹⁸ C. H. Kwon, X. Zhu, J. Zhang et al., Proceedings of the National Academy of Sciences of the United States of America 100 (22), 12923 (2003).
¹⁹ C. Settembre, A. Fraldi, L. Jahreiss et al., Human molecular genetics 17 (1), 119 (2008).
²⁰ M. Martinez-Vicente, Z. Talloczy, S. Kaushik et al., J Clin Invest (2008).

What is claimed is:

1. A method for treating a neurodegenerative disease or condition in a subject, the method comprising administering to the subject an effective amount of a compound that activates a phosphoinositide-3 kinase (PI3K) pathway, wherein the compound comprises one or more compounds selected from the group consisting of: an inhibitor of PTEN, an inhibitor of GSK3β and an activator of AKT.

2. The method of claim 1, wherein the treating comprises slowing progression of the neurodegenerative disease or condition.

3. The method of claim 1, wherein the neurodegenerative disease comprises Parkinson's disease, Alzheimer's disease or amyotrophic lateral sclerosis.

4. The method of claim 3, wherein the Parkinson's disease comprises mutation of a LRRK2 protein in the subject.

5. The method of claim 1, wherein the neurodegenerative disease or disorder comprises deficient autophagy in neurons of the subject.

6. The method of claim 1, wherein the administering comprises direct administration to the subject's brain.

7. The method of claim 1, wherein the inhibitor of PTEN comprises a vanadium complex.

8. The method of claim 1, wherein the inhibitor of PTEN comprises one or more compounds selected from the group consisting of: VO-OHpic, bpV-OHpic, pbV-pic, VO-pic, bpV-biguan, VO-biguan, bpV-phen and bpV-isoqu.

9. The method of claim 1, wherein the inhibitor of GSK3β comprises an ion, an arylindolemaleimide, a thiazole, a bis-indole, a benzazepinone or an aminopyrimidine.

10. The method of claim 1, wherein the inhibitor of GSK3β comprises one or more compounds selected from the group consisting of: indirubin-3′-monoxime, alsterpaullone, kenpaullone, SB216763, AR-A014418, CHIR98014 and lithium chloride.

11. The method of claim 1, wherein the activator of AKT comprises a plasmid capable of expressing an AKT protein, or a fragment thereof, from a nucleic acid encoding the AKT protein, or fragment thereof.
